SEQUENCE LISTING (44 sequences)

SEQ ID NO: 1 - protein, 5 aa, Artificial Sequence
  Description of Artificial Sequence Synthetic peptide
NYGMN

SEQ ID NO: 2 - protein, 17 aa, Artificial Sequence
  Description of Artificial Sequence Synthetic peptide
WINTYTREPTYADDFKG

SEQ ID NO: 3 - protein, 12 aa, Artificial Sequence
  Description of Artificial Sequence Synthetic peptide
DITAVVPTGFDY

SEQ ID NO: 4 - protein, 11 aa, Artificial Sequence
  Description of Artificial Sequence Synthetic peptide
RASENIYSNLA

SEQ ID NO: 5 - protein, 7 aa, Artificial Sequence
  Description of Artificial Sequence Synthetic peptide
AASNLAD

SEQ ID NO: 6 - protein, 9 aa, Artificial Sequence
  Description of Artificial Sequence Synthetic peptide
QHFWTTPWA

SEQ ID NO: 7 - protein, 16 aa, Artificial Sequence
  Description of Artificial Sequence Synthetic peptide
RSSQSLVHRNGNTYLH

SEQ ID NO: 8 - protein, 7 aa, Artificial Sequence
  Description of Artificial Sequence Synthetic peptide
TVSNRFS

SEQ ID NO: 9 - protein, 9 aa, Artificial Sequence
  Description of Artificial Sequence Synthetic peptide
SQSSHVPPT

SEQ ID NO: 10 - protein, 5 aa, Artificial Sequence
  Description of Artificial Sequence Synthetic peptide
NYGVN

SEQ ID NO: 11 - protein, 17 aa, Artificial Sequence
  Description of Artificial Sequence Synthetic peptide
WINPNTGEPTFDDDFKG

SEQ ID NO: 12 - protein, 11 aa, Artificial Sequence
  Description of Artificial Sequence Synthetic peptide
SRGKNEAWFAY

SEQ ID NO: 13 - protein, 9 aa, Artificial Sequence
  Description of Artificial Sequence Synthetic peptide
CVLTTLNGV

SEQ ID NO: 14 - protein, 26 aa, Artificial Sequence
  Description of Artificial Sequence Synthetic peptide
KFAHYRFTCVLTTLNGVSKKNIVVLK

SEQ ID NO: 15 - protein, 15 aa, Artificial Sequence
  Description of Artificial Sequence Synthetic peptide
HYRFTCVLTTLNGVS

SEQ ID NO: 16 - protein, 9 aa, Artificial Sequence
  Description of Artificial Sequence Synthetic peptide
CVLTTLNGV

SEQ ID NO: 17 - protein, 9 aa, Artificial Sequence
  Description of Artificial Sequence Synthetic peptide
GVSKKNIWL

SEQ ID NO: 18 - protein, 9 aa, Artificial Sequence
  Description of Artificial Sequence Synthetic peptide
ALKDLMSSV

SEQ ID NO: 19 - protein, 15 aa, Artificial Sequence
  Description of Artificial Sequence Synthetic peptide
QYIKANAKFIGITEL

SEQ ID NO: 20 - protein, 15 aa, Artificial Sequence
  Description of Artificial Sequence Synthetic peptide
GTLFPGDDDLAIPAT

SEQ ID NO: 21 - protein, 25 aa, Artificial Sequence
  Description of Artificial Sequence Synthetic peptide
HDDGLIIISIFLQVLDHKNVYFQKI

SEQ ID NO: 22 - protein, 15 aa, Artificial Sequence
  Description of Artificial Sequence Synthetic peptide
SIFLQVLDHKNVYFQ

SEQ ID NO: 23 - protein, 126 aa, Artificial Sequence
  Description of Artificial Sequence Synthetic polypeptide
MRILFLIAFMYGCVHSYVNAVETKCPNLDIVTSSGEFYCSGCVEHMSKFSYMYWLAKDMKSDEYTKFIEHLGDGIKEDETIRTTDGGITTLRKVLHVTDTNKFAHYRFTCVLTTLNGVSKKNIWLK

SEQ ID NO: 24 - protein, 44 aa, Artificial Sequence
  Description of Artificial Sequence Synthetic polypeptide
SHIQIPPGLTELLQGYTVEVLRQQPPDLVEFAVEYFTRLREARA

SEQ ID NO: 25 - protein, 45 aa, Artificial Sequence
  Description of Artificial Sequence Synthetic polypeptide
CGHIQIPPGLTELLQGYTVEVLRQQPPDLVEFAVEYFTRLREARA

SEQ ID NO: 26 - protein, 17 aa, Artificial Sequence
  Description of Artificial Sequence Synthetic peptide
QIEYLAKQIVDNAIQQA

SEQ ID NO: 27 - protein, 21 aa, Artificial Sequence
  Description of Artificial Sequence Synthetic peptide
CGQIEYLAKQIVDNAIQQAGC

SEQ ID NO: 28 - protein, 25 aa, Artificial Sequence
  Description of Artificial Sequence Synthetic peptide
GTLFPGDDDLAIPATEFFSTKAAKK

SEQ ID NO: 29 - protein, 9 aa, Artificial Sequence
  Description of Artificial Sequence Synthetic peptide
TLFPGDDDL

SEQ ID NO: 30 - protein, 25 aa, Artificial Sequence
  Description of Artificial Sequence Synthetic peptide
MKTISVVTLLCVLPAVVYSTCTVPT

SEQ ID NO: 31 - protein, 15 aa, Artificial Sequence
  Description of Artificial Sequence Synthetic peptide
ISVVTLLCVLPAVVY

SEQ ID NO: 32 - protein, 9 aa, Artificial Sequence
  Description of Artificial Sequence Synthetic peptide
TLLCVLPAV

SEQ ID NO: 33 - protein, 9 aa, Artificial Sequence
  Description of Artificial Sequence Synthetic peptide
IISIFLQVL

SEQ ID NO: 34 - protein, 40 aa, Artificial Sequence
  Description of Artificial Sequence Synthetic polypeptide
SIFLQVLDHKNVYFQGGGSCGQIEYLAKQIVDNAIQQAGC

SEQ ID NO: 35 - protein, 40 aa, Artificial Sequence
  Description of Artificial Sequence Synthetic polypeptide
CGQIEYLAKQIVDNAIQQAGCGGGSSIFLQVLDHKNVYFQ

SEQ ID NO: 36 - protein, 40 aa, Artificial Sequence
  Description of Artificial Sequence Synthetic polypeptide
HYRFTCVLTTLNGVSGGGSCGQIEYLAKQIVDNAIQQAGC

SEQ ID NO: 37 - protein, 40 aa, Artificial Sequence
  Description of Artificial Sequence Synthetic polypeptide
CGQIEYLAKQIVDNAIQQAGCGGGSHYRFTCVLTTLNGVS

SEQ ID NO: 38 - protein, 40 aa, Artificial Sequence
  Description of Artificial Sequence Synthetic polypeptide
CGQIEYLAKQIVDNAIQQAGCGGGSYHQFVIDQLKLSVNF

SEQ ID NO: 39 - protein, 40 aa, Artificial Sequence
  Description of Artificial Sequence Synthetic polypeptide
CGQIEYLAKQIVDNAIQQAGCGGGSGNCTFVTYLRHLSTV

SEQ ID NO: 40 - protein, 18 aa, Artificial Sequence
  Description of Artificial Sequence Synthetic peptide
GVQFYMIVIGVIILAALF

SEQ ID NO: 41 - protein, 26 aa, Artificial Sequence
  Description of Artificial Sequence Synthetic peptide
KKKKGVQFYMIVIGVIILAALFPSEC

SEQ ID NO: 42 - protein, 31 aa, Artificial Sequence
  Description of Artificial Sequence Synthetic polypeptide
KSGTLFPGDDDLAIPATEFFSTKAAKKPSEC

SEQ ID NO: 43 - protein, 21 aa, Artificial Sequence
  Description of Artificial Sequence Synthetic peptide
KSHYRFTCVLTTLNGVSPESC

SEQ ID NO: 44 - protein, 30 aa, Artificial Sequence
  Description of Artificial Sequence Synthetic polypeptide
HDDGLIIISIFLQVLDHKNVYFQKIGGGSC

(19) United States
(12) Patent Application Publication
(10) Pub. No.: US 2011/0064754 A1
(43) Pub. Date: Mar. 17, 2011

(54) Immunoconjugates Comprising Poxvirus-Derived Peptides and Antibodies Against Antigen-Presenting Cells for Subunit-Based Poxvirus Vaccines

(75) Inventors: Alice P. Taylor, Alpharetta, VA (US); Boby Makabi-Panzu, Newark, NJ (US); David M. Goldenberg, Mendham, NJ (US)

(73) Assignee: CENTER FOR MOLECULAR MEDICINE AND IMMUNOLOGY, Belleville, NJ (US)

(21) Appl. No.: 12/915,515

(22) Filed: Oct. 29, 2010

Related U.S. Application Data
(63) Continuation-in-part of application No. 12/754,140, filed on Apr. 5, 2010.
Application No. 12/754,140 is a continuation-in-part of application No. 12/556,718, filed on Sep. 10, 2009.
Application No. 12/556,718 is a division of application No. 11/368,296, filed on Mar. 3, 2006, now Pat. No. 7,612,180.
Application No. 11/368,296 is a continuation-in-part of application No. 12/754,740, filed on Apr. 6, 2010.
Application No. 12/754,740 is a continuation-in-part of application No. 12/544,476, filed on Aug. 20, 2009.
(60) Provisional application No. 60/657,695, filed on Mar. 3, 2005.
(60) Provisional application No. 61/166,809, filed on Apr. 6, 2009.
(60) Provisional application No. 61/168,715, filed on Apr. 13, 2009.
(60) Provisional application No. 61/090,487, filed on Aug. 20, 2008.
(60) Provisional application No. 61/168,290, filed on Apr. 10, 2009.
(60) Provisional application No. 61/258,369, filed on Nov. 5, 2009.
(60) Provisional application No. 61/258,729, filed on Nov. 6, 2009.
(60) Provisional application No. 61/378,059, filed on Aug. 30, 2010.

(51) Int. Cl. A61K 39/395; C07K 16/08; A61K 39/285; A61P 31/20; A61P 37/04

(52) U.S. Cl. 424/178.1; 530/391.1; 424/196.11

(57) ABSTRACT

The present invention concerns methods and compositions for subunit-based vaccines for inducing immunity against poxvirus infections, such as smallpox. Preferred embodiments concern immunoconjugates comprising one or more subunit antigenic peptides attached to an antibody or fragment thereof that targets antigen-producing cells (APCs). More preferably, the antibody binds to HLA-DR and the antigenic peptide is from an immunomodulating factor, such as the viral IL-18 binding protein (vIL18BP). However, mixtures of antigenic peptides from different viral proteins may also be used. The vaccine is capable of inducing immunity against poxvirus without risk of disseminated infection in immunocompromised hosts or transmission to susceptible contacts.

### RELATED APPLICATIONS

This application is a continuation-in-part of U.S. patent application Ser. No. 12/754,140, filed Apr. 5, 2010, which was a continuation-in-part of U.S. patent application Ser. No. 12/556,718, filed Sep. 10, 2009, which was a divisional of U.S. Pat. No. 7,612,180, filed Mar. 3, 2006. Those applications claimed the benefit under 35 USC 119(e) of provisional U.S. Patent Applications 61/166,809, filed Apr. 6, 2009; 61/168,715, filed Apr. 13, 2009; and 60/657,695 filed on Mar. 3, 2005. This application is a continuation-in-part of U.S. patent application Ser. No. 12/754,740, filed Apr. 6, 2010, which was a continuation-in-part of U.S. patent application Ser. No. 12/544,476, filed Aug. 20, 2009, which claimed the benefit under 35 USC 119(e) of provisional U.S. Patent Applications 61/090,487, filed Aug. 20, 2008, and 61/168,290, filed Apr. 10, 2009. This application claims the benefit under 35 USC 119(e) of provisional U.S. Patent Applications 61/258,369, filed Nov. 5, 2009; 61/258,729, filed Nov. 6, 2009; and 61/378,059, filed Aug. 30, 2010. The text of each priority application is incorporated herein by reference in its entirety.

### BACKGROUND

1. Field of the Invention

The present invention relates to the design, generation and use of subunit-based vaccines for the treatment and/or prevention of poxvirus infections, including but not limited to smallpox. In preferred embodiments, the vaccines comprise an immunoconjugate of a subunit antigenic peptide derived from one or more viral proteins. In more preferred embodiments the viral proteins are immunomodulating factors, such as the viral IL-18 binding protein (vIL18BP), although alternative viral proteins may be used, such as viral envelope proteins. In other alternative embodiments, subunit-based vaccines may comprise combinations of antigenic peptides from more than one viral protein, such as an immunomodulating factor and an envelope protein. The viral antigenic peptide is attached to an antibody or antigen-binding fragment thereof that targets the subunit to antigen-producing cells (APCs). In most preferred embodiments, the subunit-based vaccine incorporates an antibody or antibody fragment against the HLA-DR antigen, such as the L243 antibody; although the skilled artisan will realize that other APC targeting antibodies are known and may be used. Use of the immunoconjugate provides substantially increased immunogenicity and improved immune system response against viral antigens, while avoiding the possibility of infection of immunocompromised individuals exposed to live virus-based vaccines. Preferably, the subunit-based vaccine is effective to induce immunity against and to prevent infection by smallpox and/or other poxviruses in vivo.

2. Related Art

The Orthopoxviruses, a group of complex viruses with cross-reacting antigens, includes vaccinia virus (VV), monkeypox virus, and the virus that causes smallpox (variola, VAR). Smallpox is no longer a naturally occurring infection, having been eradicated by a massive immunization program up to 1978, when routine vaccination of the world's population ceased (Minor, 2002, British Med J 62:213-224). At that time, the remaining stocks of virus were deposited in the U.S. and the former Soviet Union. The recent threat of bioterrorism, a recent outbreak of monkeypox (CDC MMWR 2003), and the deadly nature of smallpox disease, especially in a world where it is estimated that less than half the population is vaccinated, has stimulated renewed interest in development of vaccine protection against VAR, or other dangerous members of this family of viruses.

Smallpox vaccine, which employs active VV, currently represents the most effective means to immunize against smallpox (Rosenthal et al, 2001, Emerg. Infect. Dis. 7: 920-926). This vaccine produces a transient viremia which is resolved in most individuals, and which leaves long-lasting immunity. However, this vaccine also raises safety issues because of serious adverse reactions, which include systemic viremia and death (He et al, 2007, JID 196: 1026-1032; Rosenthal et al, 2001). Therefore, development of alternative vaccination strategies is required if circumstances necessitate immunization of the population.

Several approaches to alternative vaccines have been tried, or are in development. Attenuated forms of poxvirus, such as the Akhara Modified Vaccinia (MVA), with deleted or mutated genes (Grandpre et al., Vaccine 27:1549-56, 2009), may confer partial immunity to highly virulent strains of poxvirus. Immunization with inactivated virus has been investigated, but it does not confer the same degree of protection as live virus; 10³-10⁴ more units of inactivated virus are required to protect mice from challenge, regardless of the inactivation method used (Turner et al. 1970, J. Hyg. Camb 68:197-210). This fact implies that the protection derived from immunization with active virus includes factors that are not produced by, or are not present in, inactive virus. Poxviruses produce a spectrum of secreted host immune response modifying factors which neutralize host cytokines and innate defense mechanisms. By weakening the host's first line of defense, the virus may be able to establish infection (e.g. in the mucosa) and begin the first phase of infectious replication in host cells.

A need exists for vaccines against poxviruses, such as smallpox, that are more effective than inactivated virus but which avoid the safety issues seen with live virus vaccines.

### SUMMARY OF THE INVENTION

The present invention discloses improved compositions and methods of use of subunit vaccines against poxviruses, such as smallpox. In preferred embodiments, the vaccine comprises one or more subunit antigenic peptides conjugated to an antibody or antibody fragment that binds to antigen presenting cells (APCs), such as dendritic cells (DCs), to form an immunoconjugate. Administration of the vaccine to subjects induces immunity against the poxvirus and is effective to treat or prevent poxvirus infection. Optionally, the vaccine may incorporate one or more adjuvants, such as aluminum hydroxide, CpG DNA, calcium phosphate or bacterial-based adjuvant (e.g., L. delbroeckii/bulgaricus).

One host immune modulating factor produced by both VV and VAR is the viral interleukin-18 binding protein (vIL18BP, vaccinia virus C12L gene). Like other viral host defense modulating factors, this gene is expressed in the early phase of infection, and it cripples host immunity by neutralizing a key pro-inflammatory cytokine, IL-18, which stimulates NK, CD8, and Th1 CD4 cells to produce interferon-gamma (IFN), which in turn activates antigen presenting cells (APCs), and other cells and which directs immune responses toward the Th1 type (Born et al, 2000, J. Immunol. 164: 3246-3254; Scott, 1991, J Immunol 147:3149-3155; Pien et al, 2002, Immunol. 169:5827-5837; Xiang and Moss, 1999, Proc. Natl. Acad. Sci. USA 96:11537-11542). In preferred embodiments, the subunit antigenic peptide is selected to mimic an epitope of vIL18BP. Other exemplary host immune modulating factors and their locus tag identifiers are provided in Table 1.

In other preferred embodiments the subunit antigenic peptide is derived from a viral immunomodulating protein. The skilled artisan will realize that various viral immunomodulating proteins are known and may be of use. Non-limiting examples include the interferon-gamma (IFN-gamma) receptor homolog (B8R gene), complement control protein homolog (B5R gene) and serine protease inhibitors (B13R, B14R, B22R genes). A wide variety of poxvirus immunomodulatory proteins have been reported, although their effect on viral immunogenicity has not been well characterized. (See, e.g., Jackson et al. J Virol 79:6554-59, 2005; B12R gene (ser/thr protein kinase); B15R gene (IL-1 and IL-6 receptor); B16R gene (IL-1 receptor), B18R gene (IFN-α receptor), B19R gene (IL-1 and IL-6 receptor, IFN inhibitor.)

TABLE 1
Poxvirus Immunomodulating Proteins

| | |
|---|---|
| VACWR001 | TNF-alpha receptor-like |
| VACWR011 | apoptosis |
| VACWR012 | zinc(Zn)-finger-like |
| VACWR013 (VAC WR C12L) | IL-18 binding protein |
| VACWR025 | blocks C3b/C4b complement |
| | activation |
| VACWR028 | intracellular signal transduction |
| | inhibitor |
| VACWR033 | serine protease inhibitor |
| VACWR034 | interferon resistance |
| VACWR059 | ds RNA binder; interferon binder |
| VACWR172 | Toll-like receptor modulator |
| VACWR190 | IFN-gamma receptor-like |
| VACWR208 | Zn-finger-like |
| VACV WR 215 | TNF-alpha R |
| VACWR217 | TNF-alpha receptor-like |
| VACV COP B19R (VACWR200) | IFN-type I binder |
| VACV COP A39R | semaphoring-like |
| VACV COP A40R | type II membrane protein |
| VACV COP A41L | secreted glycoprotein |
| VACV A4L | immunodominant antigen |
| VACV A27L (VACWR150) | surface binding heparin sulfate |
| VACV D8L (VACWR113) | surface binding chondroitin sulfate |
| VACV B5R (VACWR187) | essential for membrane |
| | wrapping of IMV in trans-Golgi |

In alternative embodiments, the subunit antigenic peptide may be derived from a viral envelope protein or other viral proteins. Non-limiting examples include the protein products of the D8L, A27L, L1R and A33R genes. The skilled artisan will realize that the DNA and amino acid sequences of the various poxyiral genes and proteins are well known in the art and are publicly available (see, e.g., GenBank Accession No. AY243312 for the complete genomic sequence of Vaccinia virus WR, along with the encoded protein sequences).

The antibody component of the immunoconjugate directs the complex to APCs, where the antigenic peptide component is processed to invoke an immune response against poxviruses and/or infected cells expressing the target antigen. Various APC targeting antibodies are known in the art, such as antibodies that bind to an antigen selected from the group consisting of HLA-DR, CD74, CD209 (DC-SIGN), CD34, CD74, CD205, TLR 2 (toll-like receptor 2), TLR 4, TLR 7, TLR 9, BDCA-2, BDCA-3 and BDCA-4. In more preferred embodiments, the antibody binds to an antigen selected from HLA-DR and CD74. In most preferred embodiments, the antibody binds to HLA-DR.

In certain preferred embodiments, the poxvirus vaccine comprises a humanized, human or chimeric anti-HLA-DR antibody, such as the L243 antibody. The L243 antibody has been described (e.g., U.S. Pat. No. 7,612,180, the Examples section of which is incorporated herein by reference) and is characterized by having heavy chain complementarity determining region (CDR) sequences CDR1 (NYGMN, SEQ ID NO:1), CDR2 (WINTYTREPTYADDFKG, SEQ ID NO:2), and CDR3 (DITAVVPTGFDY, SEQ ID NO:3) and light chain CDR sequences CDR1 (RASENIYSNLA, SEQ ID NO:4), CDR2 (AASNLAD, SEQ ID NO:5), and CDR3 (OHFWTTPWA, SEQ ID NO:6). However, other anti-HLA-DR antibodies known in the art may be used (see, e.g., U.S. Pat. Nos. 6,416,958, 6,894,149; 7,262,278, the Examples section of each of which is incorporated herein by reference).

In other preferred embodiments, the poxvirus vaccine comprises a humanized, human or chimeric anti-CD74 antibody, such as the LL1 antibody. The LL1 antibody has been described (e.g., U.S. Pat. No. 7,312,318, the Examples section of which is incorporated herein by reference) and is characterized by having light chain CDR sequences CDR1 (RSSQSLVHRNGNTYLH; SEQ ID NO:7), CDR2 (TVSNRFS; SEQ ID NO:8), and CDR3 (SQSSHVPPT; SEQ ID NO:9) and heavy chain CDR sequences CDR1 (NYGVN; SEQ ID NO:10), CDR2 (WINPNTGEPTFDDDFKG; SEQ ID NO:11), and CDR3 (SRGKNEAWFAY; SEQ ID NO:12). Alternatively, other anti-CD74 antibodies or antibodies against other APC- or DC-associated antigens may be utilized (see, e.g., LifeSpan Biosciences Inc., Seattle, Wash.; BioLegend, San Diego, Calif.; Abcam, Cambridge, Mass.).

In various embodiments, the antibody or antigen-binding fragment thereof may be chimeric, humanized or human. The use of chimeric antibodies is preferred to the parent murine antibodies because they possess human antibody constant region sequences and therefore do not elicit as strong a human anti-mouse antibody (HAMA) response as murine antibodies. The use of humanized antibodies is even more preferred, in order to further reduce the possibility of inducing a HAMA reaction. As discussed below, techniques for humanization of murine antibodies by replacing murine framework and constant region sequences with corresponding human antibody framework and constant region sequences are well known in the art and have been applied to numerous murine anti-cancer antibodies. Antibody humanization may also involve the substitution of one or more human framework amino acid residues with the corresponding residues from the parent murine framework region sequences. As also discussed below, techniques for production of human antibodies are also well known and such antibodies may be incorporated into the subject poxvirus vaccine constructs.

Still other embodiments relate to DNA sequences encoding fusion proteins, such as antibody-subunit antigenic peptide fusion proteins, vectors and host cells containing the DNA sequences, and methods of making fusion proteins for the production of poxvirus vaccines. In certain embodiments, where DNL (dock-and-lock) technology is used to make the subunit vaccine, the fusion proteins may comprise DDD (dimerization and docking domain) moieties or AD (anchoring domain) moieties. In alternative embodiments, the immunoconjugate may be formed by chemical cross-linking of, for example, an antibody or antibody fragment and an antigenic peptide.

### DETAILED DESCRIPTION OF THE INVENTION

### Definitions

As used herein, the terms “a”, “an” and “the” may refer to either the singular or plural, unless the context otherwise makes clear that only the singular is meant.

As used herein, the term “about” means plus or minus ten percent (10%) of a value. For example, “about 100” would refer to any number between 90 and 110.

An antibody refers to a full-length (i.e., naturally occurring or formed by normal immunoglobulin gene fragment recombinatorial processes) immunoglobulin molecule (e.g., an IgG antibody) or an immunologically active, antigen-binding portion of an immunoglobulin molecule, like an antibody fragment.

An antibody fragment is a portion of an antibody such as F(ab′)₂, F(ab)₂, Fab′, Fab, Fv, scFv and the like. Regardless of structure, an antibody fragment binds with the same antigen that is recognized by the intact antibody. Therefore the term is used synonymously with “antigen-binding antibody fragment.” The term “antibody fragment” also includes isolated fragments consisting of the variable regions, such as the “Fv” fragments consisting of the variable regions of the heavy and light chains and recombinant single chain polypeptide molecules in which light and heavy variable regions are connected by a peptide linker (“scFv proteins”). As used herein, the term “antibody fragment” does not include portions of antibodies without antigen binding activity, such as Fc fragments or single amino acid residues. Other antibody fragments, for example single domain antibody fragments, are known in the art and may be used in the claimed constructs. (See, e.g., Muyldermans et al., TIBS 26:230-235, 2001; Yau et al., J Immunol Methods 281:161-75, 2003; Maass et al., J Immunol Methods 324:13-25, 2007).

The term antibody fusion protein may refer to a recombinantly produced antigen-binding molecule in which one or more of the same or different single-chain antibody or antibody fragment segments with the same or different specificities are linked. Valency of the fusion protein indicates how many binding arms or sites the fusion protein has to a single antigen or epitope; i.e., monovalent, bivalent, trivalent or multivalent. The multivalency of the antibody fusion protein means that it can take advantage of multiple interactions in binding to an antigen, thus increasing the avidity of binding to the antigen. Specificity indicates how many antigens or epitopes an antibody fusion protein is able to bind; i.e., monospecific, bispecific, trispecific, multispecific. Using these definitions, a natural antibody, e.g., an IgG, is bivalent because it has two binding arms but is monospecific because it binds to one epitope. Monospecific, multivalent fusion proteins have more than one binding site for an epitope but only bind with one epitope. The fusion protein may comprise a single antibody component, a multivalent or multispecific combination of different antibody components or multiple copies of the same antibody component. The fusion protein may additionally comprise an antibody or an antibody fragment and a subunit peptide antigen. However, the term is not limiting and a variety of protein or peptide effectors may be incorporated into a fusion protein. In another non-limiting example, a fusion protein may comprise an AD or DDD sequence for producing a DNL construct as discussed below.

A chimeric antibody is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A humanized antibody is a recombinant protein in which the CDRs from an antibody from one species; e.g., a rodent antibody, are transferred from the heavy and light variable chains of the rodent antibody into human heavy and light variable domains (e.g., framework region sequences). The constant domains of the antibody molecule are derived from those of a human antibody. In certain embodiments, a limited number of framework region amino acid residues from the parent (rodent) antibody may be substituted into the human antibody framework region sequences.

A human antibody is, e.g., an antibody obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous murine heavy chain and light chain loci. The transgenic mice can synthesize human antibodies specific for particular antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. See for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for review, see e.g. Johnson and Chiswell, Current Opinion in Structural Biology 3:5564-571 (1993). Human antibodies may also be generated by in vitro activated B cells. See U.S. Pat. Nos. 5,567,610 and 5,229,275, the Examples sections of which are incorporated herein by reference.

### BRIEF DESCRIPTION OF THE DRAWINGS

FIG. 1. Binding and uptake of peptides derived from vIL18BP sequence (SEQ ID NO:23). (A) vIL18BP110 (SEQ ID NO:16) bound to T2 cells. Indicated peptides (TT830, SEQ ID NO:19; vA4L229, SEQ ID NO:18; vIL18BP008, SEQ ID NO:13; vIL18BP105, SEQ ID NO:15; vIL18BP110, SEQ ID NO:16; vIL18BP117, SEQ ID NO:17) were incubated with T2 cells for 24 h. Relative abundance of HLA-A02 on T2 cells is shown. Each bar, left to right, represents increasing concentrations of peptide from 0 to 40 μg/mL in 10-μg/mL increments. (B) vIL18BP105 (SEQ ID NO:15) demonstrated the highest uptake by donor PBMCs. Duplicate samples were evaluated after incubation with the indicated biotinylated peptides for 24 h. NJ01, NJ04, NJ07 and NJ08. Results were analyzed by flow cytometry after addition of an avidin-FITC conjugate. Fluorescence value for each peptide equals fluorescence value of peptide-treated cells minus the fluorescence value of untreated cells in the same experiment. Peptide concentration was 20 μg/mL.

FIG. 2. PBMCs from vaccinated donors proliferate when incubated with viral peptides. CFSE-loaded PBMCs from vaccinated (A) and unvaccinated (B) human donors were incubated with 10 μg/mL of the designated peptide (vA27L003, SEQ ID NO:20; vD8L118, SEQ ID NO:22; vIL18BP105, SEQ ID NO:15 or control) for 5 days. Cells were harvested and analyzed by flow cytometry (means±SD). Bars shown in order: open bars, medium control; solid black bars, 2.5 mg/mL peptide (or SEA); horizontal-hatch light-grey bars, 5.0 mg/mL peptide; vertical-hatch dark-grey bars, 10.0 mg/mL peptide. (C) Results from separate experiments where cells from the designated samples were incubated with vD8L118 (SEQ ID NO:22) to determine intracellular cytokine and activation marker expression. The results are shown in the embedded table (C) (D8L, vD8L118 peptide, SEQ ID NO:22). *Group average P<0.05 vs. medium controls (t-test).

FIG. 3. The responding CD8+ IFN-γ+ cells have the phenotype of T_{EM} or CD45RA-terminally differentiated cells. CD8+ PBMCs from vaccinated donors were assessed for CD45RA and CCR7 expression. The numbers represent percentage of total cells. P<0.019 vD8L118 vs. medium controls (ANOVA) for the T_{EM} population (lower left quadrant).

FIG. 4. CD107a expression by CD8+ cells. CD8+ cell population was assessed for IFN-γ, IL-2 vs. degranulation potential marker CD107a. Numbers and bar values represent percentage of gated cells for (A) CD8+IFN-γ+ cells, (B) CD8+IFN-γ− cells, and (C) CD8+IL-2+ cells. * group average P<0.04 vs. medium control (t-test).

FIG. 5. Antibody to peptides is present in serum from vaccinated donors. Serum from unvaccinated or vaccinated donors was diluted 1:200 and incubated with peptide immobilized on 96-well plates in a modified ELISA for (A) peptide vA27L003 (SEQ ID NO:20), (B) peptide vD8L110 (SEQ ID NO:21), and (C) peptide vIL18BP102 (SEQ ID NO:14). Dots represent the A450 for each donor. *P<0.03 vs. unvaccinated (ANOVA). Unvaccinated donors: 213, 704, 220; vaccinated donors: 05, 12A, 12B, 19, 26, 720, 308, 416, and 920. Peptides vD8L110 and vIL18BP105 were 25-mers which included the full sequences of vD8L118 and vIL18BP105.

FIG. 6. HLA-DR04 tg splenocyte proliferation to vIL18BP105. HLA-DR04 tg mice were immunized with vIL18BP105-L243 conjugate (conj) or free vIL18BP105 (Free), naïve HLA-DR04 tg mice (HLA-DR04 tg naive) and wild type C57BL/6J (WT naive) (n=3 mice/group). Assays were performed in triplicate with CFSE-labeled splenocytes incubated with varied concentrations of peptides. Results are typical of 3 separate experiments (n=3, means±SD).

FIG. 7. Peptide-specific serum antibody production in HLA-DR04 tg mice immunized with CIL18BP105 (Conjugate) and IL18BP105 (Free) 7 and 14 days following the first boost. Naïve HLA-DR04 tg mouse serum was used as control (Naïve). Experiments were performed in triplicate with pooled sera (n=3, means±SD).

FIG. 8. Binding of serum antibodies from immunized mice to intact vIL18BP protein. Serum from mice immunized with L243 antibody alone, vIL18BP105 peptide (SEQ ID NO:15), the viral IL18BP105 peptide conjugated to L243 antibody (CIL18BP105), medium alone, or naive mice was tested for antibodies recognizing intact vIL18BP protein.

FIG. 9. Liposome based immunoconjugate for subunit vaccine. (A) Liposome-displayed peptide-L243 antibody conjugate. (B) Liposome-displayed bare peptide without antibody.

### POXVIRUS VACCINES

### Subunit Antigenic Peptides

Poxviruses produce a spectrum of secreted host immune-response modifying factors which neutralize host cytokines and innate defense mechanisms. Weakening the host's first line of defense allows the virus to establish infection (e.g., in the mucosa) and then begin the first phase of infectious replication. One factor produced by VV and other poxviruses is the viral interleukin-18 binding protein (vIL18BP, vaccinia virus C12L gene), expressed in the early phase of infection (Born et al. J Immunol 164:3246-54, 2000). It works by neutralizing a key pro-inflammatory cytokine, IL-18, which stimulates NK, CD8, and Th1 CD4 cells to produce interferon-γ (IFN-γ), which directs acquired immunity toward the Th1 type (Livingston et al., J Immunol 168:5499-5506, 2002; Pien et al., J Immunol 160:5827-37, 2002; Scott, J Immunol 147:3149-55, 1991; Turner et al., J Hyg Camb 68:197-210, 1970; Xiang and Moss, PNAS USA 96:11537-542, 1999).

The studies described in the Examples below were addressed to the question of whether or not host response against vIL18BP is involved in resistance to poxvirus infection. If so, an alternative vaccine strategy should include this factor and/or similar antigens. It has recently been reported that another orthopoxvirus host defense-modulating factor, type-I IFN-binding protein, was essential for virulence (Xu et al., J Exp Med 205:981-92, 2008) and may be a candidate for inclusion in a subunit poxvirus vaccine. As discussed below, other known viral proteins may also be candidates for inclusion as subunit antigenic peptides for a subunit-based poxvirus vaccine.

As described in the following Examples, the subunit peptide-based vaccine approach to human immunity was tested by investigating whether vIL18BP antigen peptides were able to elicit recall responses by peripheral blood mononuclear cells (PBMCs), and serum of vaccinated human subjects. The importance of cell-mediated immunity in resistance to poxvirus remains under investigation, but it is fairly well established that antibody response is required for immunity (Chaudhri et al., J Virol 80:6339-44, 2006; Combadiere et al., J Exp Med 199, 1585-89, 2004; Kim et al., Clin Vaccine Immunol 13:1172-74, 2006).

In addition to the vIL18BP-derived peptides, peptides derived from other VV genes, D8L and A27L, were also tested. The D8L protein is important for viral attachment and entry into cells, and has been shown to elicit strong protective immunity in mouse models of poxvirus infection (Kan-Mitchell et al., J Immunol 172:5249-61, 2010; Berhanu et al., J Virol 82:3517-29, 2008). The A27L protein is also important for viral attachment and assembly, and antibodies against it provide protective immunity (Berhanu et al., J Virol 82:3517-29, 2008; Chun g et al., J Virol 72:1577-85, 1998; Scott, J Immunol 147:3149-55, 1991).

The overall goal of this invention was to select and develop T-cell (HLA-binding) and B-cell antigen peptides for inclusion in a multi-epitopic vaccine format. Peptides have the advantages of being relatively easy to synthesize, modify, and combine into multi-antigen complexes. To enhance their immunogenicity, the peptides were attached to antibodies targeting APCs, such as antibodies against the HLA-DR antigen.

APC-Targeting Antibodies

As the professional antigen-presenting cells, dendritic cells (DCs) play a pivotal role in orchestrating innate and adaptive immunity, and have been harnessed to create effective vaccines (Vulink et al., Adv Cancer Res. 2008, 99:363-407; O'Neill et al., Mol. Biotechnol. 2007, 36:131-41). In vivo targeting of antigens to APCs and DCs represents a promising approach for vaccination, as it can bypass the laborious and expensive ex vivo antigen loading and culturing, and facilitate large-scale application of immunotherapy (Tacken et al., Nat Rev Immunol. 2007, 7:790-802). More significantly, in vivo APC and/or DC targeting vaccination is more efficient in eliciting anti-tumor immune response, and more effective in controlling tumor growth in animal models (Kretz-Rommel et al., J Immunother 2007, 30:715-726).

In addition to DCs, B cells are another type of potent antigen-presenting cells capable of priming Th1/Th2 cells (Morris et al, J. Immunol. 1994, 152:3777-3785; Constant, J. Immunol. 1999, 162:5695-5703) and activating CD8 T cells via cross-presentation (Heit et al., J. Immunol. 2004, 172:1501-1507; Yan et al., Int Immunol. 2005, 17:869-773). It was recently reported that in vivo targeting of antigens to B cells breaks immune tolerance of MUC1 (Ding et al., Blood 2008, 112:2817-25).

In various embodiments of the present invention, antibodies against antigens expressed by APCs in general and DCs in particular may be incorporated into immunoconjugate vaccines to target subunit antigenic peptides to immune system cells. Two exemplary APC antigens are HLA-DR and CD74. HLA-DR is a major histocompatibility complex class II cell surface receptor which functions in antigen presentation to elicit T-cell immune responses. HLA-DR is found on a wide variety of antigen presenting cells, such as macrophages, B-cells and dendritic cells. As discussed above, antibodies against HLA-DR, including the L243 antibody, are known in the art. Such antibodies may be conjugated to subunit antigenic peptides for delivery to APCs.

Another APC expressed antigen is CD74, which is a type II integral membrane protein essential for proper MHC II folding and targeting of MHC II-CD74 complex to the endosomes (Stein et al., Clin Cancer Res. 2007, 13:5556s-5563s; Matza et al., Trends Immunol. 2003, 24(5):264-8). CD74 expression is not restricted to DCs, but is found in almost all antigen-presenting cells (Freudenthal et al., Proc Natl Acad Sci USA. 1990, 87:7698-702; Clark et al., J. Immunol. 1992, 148(11):3327-35). The wide expression of CD74 in APCs may offer some advantages over sole expression in myeloid DCs, as targeting of antigens to other APCs like B cells has been reported to break immune tolerance (Ding et al., Blood 2008, 112:2817-25), and targeting to plasmacytoid DCs cross-presents antigens to naive CD8 T cells. More importantly, CD74 is also expressed in follicular DCs (Clark et al., J. Immunol. 1992, 148(11):3327-35), a DC subset critical for antigen presentation to B cells (Tew et al., Immunol Rev. 1997, 156:39-52). This expression profile makes CD74 an excellent candidate for in vivo targeting vaccination. A variety of anti-CD74 antibodies are known in the art, such as the LL1 antibody (Leung et al., Mol. Immunol. 1995, 32:1416-1427; Losman et al., Cancer 1997, 80:2660-2666; Stein et al., Blood 2004, 104:3705-11).

Antibodies and Antibody Fragments

In various embodiments, antibodies or antigen-binding fragments of antibodies may be incorporated into the poxvirus vaccine. Antigen-binding antibody fragments are well known in the art, such as F(ab′)₂, F(ab)₂, Fab′, Fab, Fv, scFv and the like, and any such known fragment may be used. As used herein, an antigen-binding antibody fragment refers to any fragment of an antibody that binds with the same antigen that is recognized by the intact or parent antibody. Techniques for preparing conjugates of virtually any antibody or fragment of interest are known (e.g., U.S. Pat. No. 7,527,787).

Techniques for preparing monoclonal antibodies against virtually any target antigen, such as HLA-DR or CD74, are well known in the art. See, for example, Kohler and Milstein, Nature 256:495 (1975), and Coligan et al. (eds.), CURRENT PROTOCOLS IN IMMUNOLOGY, VOL. 1, pages 2.5.1-2.6.7 (John Wiley & Sons 1991). Briefly, monoclonal antibodies can be obtained by injecting mice with a composition comprising an antigen, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

MAbs can be isolated and purified from hybridoma cultures by a variety of well-established techniques. Such isolation techniques include affinity chromatography with Protein-A SEPHAROSE®, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992).

After the initial raising of antibodies to the immunogen, the antibodies can be sequenced and subsequently prepared by recombinant techniques. Humanization and chimerization of murine antibodies and antibody fragments are well known to those skilled in the art. The use of antibody components derived from humanized, chimeric or human antibodies obviates potential problems associated with the immunogenicity of murine constant regions.

Chimeric Antibodies

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. General techniques for cloning murine immunoglobulin variable domains are disclosed, for example, in Orlandi et al., Proc. Nat'l Acad. Sci. USA 86:3833 (1989). Techniques for constructing chimeric antibodies are well known to those of skill in the art. As an example, Leung et al., Hybridoma 13:469 (1994), produced an LL2 chimera by combining DNA sequences encoding the V_{κ} and V_{H} domains of murine LL2, an anti-CD22 monoclonal antibody, with respective human κ and IgG₁ constant region domains.

Humanized Antibodies

Techniques for producing humanized MAbs are well known in the art (see, e.g., Jones et al., Nature 321:522 (1986), Riechmann et al., Nature 332:323 (1988), Verhoeyen et al., Science 239:1534 (1988), Carter et al., Proc. Nat'l Acad. Sci. USA 89:4285 (1992), Sandhu, Crit. Rev. Biotech. 12:437 (1992), and Singer et al., J. Immun. 150:2844 (1993)). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al., Biotechnology 9:266 (1991) and Verhoeyen et al., Science 239:1534 (1988). Generally, those human FR amino acid residues that differ from their murine counterparts and are located close to or touching one or more CDR amino acid residues would be candidates for substitution. Humanized forms of the L243 and LL1 antibodies are known (see, e.g., U.S. Pat. Nos. 7,612,180 and 7,312,318).

Human Antibodies

Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Phamacol. 3:544-50). A fully human antibody also can be constructed by genetic or chromosomal transfection methods, as well as phage display technology, all of which are known in the art. See for example, McCafferty et al., Nature 348:552-553 (1990). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Recombinant Fab were cloned from the μ, γ and κ chain antibody repertoires and inserted into a phage display library (Id.). RNAs were converted to cDNAs and used to make Fab cDNA libraries using specific primers against the heavy and light chain immunoglobulin sequences (Marks et al., 1991, 1 Mol. Biol. 222:581-97). Library construction was performed according to Andris-Widhopf et al. (2000, In: Phage Display Laboratory Manual, Barbas et al. (eds), 1^{st} edition, Cold Spring Harbor Laboratory Press, Cold Spring Harbor, N.Y. pp. 9.1 to 9.22). The final Fab fragments were digested with restriction endonucleases and inserted into the bacteriophage genome to make the phage display library. Such libraries may be screened by standard phage display methods, as known in the art (see, e.g., Pasqualini and Ruoslahti, 1996, Nature 380:364-366; Pasqualini, 1999, The Quart. J. Nucl. Med. 43:159-162).

Phage display can be performed in a variety of formats, for their review, see e.g. Johnson and Chiswell, Current Opinion in Structural Biology 3:5564-571 (1993). Human antibodies may also be generated by in vitro activated B-cells. See U.S. Pat. Nos. 5,567,610 and 5,229,275, incorporated herein by reference in their entirety. The skilled artisan will realize that these techniques are exemplary and any known method for making and screening human antibodies or antibody fragments may be utilized.

In another alternative, transgenic animals that have been genetically engineered to produce human antibodies may be used to generate antibodies against essentially any immunogenic target, using standard immunization protocols. Methods for obtaining human antibodies from transgenic mice are disclosed by Green et al., Nature Genet. 7:13 (1994), Lonberg et al., Nature 368:856 (1994), and Taylor et al., Int. Immun. 6:579 (1994). A non-limiting example of such a system is the XenoMouse® (e.g., Green et al., 1999, J. Immunol. Methods 231:11-23) from Abgenix (Fremont, Calif.). In the XenoMouse® and similar animals, the mouse antibody genes have been inactivated and replaced by functional human antibody genes, while the remainder of the mouse immune system remains intact.

The XenoMouse® was transformed with germline-configured YACs (yeast artificial chromosomes) that contained portions of the human IgH and Igkappa loci, including the majority of the variable region sequences, along accessory genes and regulatory sequences. The human variable region repertoire may be used to generate antibody producing B-cells, which may be processed into hybridomas by known techniques. A XenoMouse® immunized with a target antigen will produce human antibodies by the normal immune response, which may be harvested and/or produced by standard techniques discussed above. A variety of strains of XenoMouse® are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the XenoMouse® system but may utilize any transgenic animal that has been genetically engineered to produce human antibodies.

Antibody Fragments

Antibody fragments which recognize specific epitopes can be generated by known techniques. Antibody fragments are antigen binding portions of an antibody, such as F(ab′)₂, Fab′, F(ab)₂, Fab, Fv, sFv and the like. F(ab′)₂ fragments can be produced by pepsin digestion of the antibody molecule and Fab′ fragments can be generated by reducing disulfide bridges of the F(ab′)₂ fragments. Alternatively, Fab′ expression libraries can be constructed (Huse et al., 1989, Science, 246:1274-1281) to allow rapid and easy identification of monoclonal Fab′ fragments with the desired specificity. F(ab)₂ fragments may be generated by papain digestion of an antibody and Fab fragments obtained by disulfide reduction.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are described in U.S. Pat. No. 4,704,692, U.S. Pat. No. 4,946,778, R. Raag and M. Whitlow, “Single Chain Fvs.” FASEB Vol 9:73-80 (1995) and R. E. Bird and B. W. Walker, “Single Chain Antibody Variable Regions,” TIBTECH, Vol 9:132-137 (1991).

Techniques for producing single domain antibodies are also known in the art, as disclosed for example in Cossins et al. (2006, Prot Express Purif 51:253-259). Single domain antibodies (VHH) may be obtained, for example, from camels, alpacas or llamas by standard immunization techniques. (See, e.g., Muyldermans et al., TIBS 26:230-235, 2001; Yau et al., J Immunol Methods 281:161-75, 2003; Maass et al., J Immunol Methods 324:13-25, 2007). The VHH may have potent antigen-binding capacity and can interact with novel epitopes that are inaccessible to conventional VH-VL pairs. (Muyldermans et al., 2001). Alpaca serum IgG contains about 50% camelid heavy chain only IgG antibodies (HCAbs) (Maass et al., 2007). Alpacas may be immunized with known antigens, such as TNF-α, and VHHs can be isolated that bind to and neutralize the target antigen (Maass et al., 2007). PCR primers that amplify virtually all alpaca VHH coding sequences have been identified and may be used to construct alpaca VHH phage display libraries, which can be used for antibody fragment isolation by standard biopanning techniques well known in the art (Maass et al., 2007).

An antibody fragment can be prepared by proteolytic hydrolysis of the full length antibody or by expression in E. coli or another host of the DNA coding for the fragment. An antibody fragment can be obtained by pepsin or papain digestion of full length antibodies by conventional methods. These methods are described, for example, by Goldenberg, U.S. Pat. Nos. 4,036,945 and 4,331,647 and references contained therein. Also, see Nisonoff et al., Arch Biochem. Biophys. 89:230 (1960); Porter, Biochem. J. 73:119 (1959), Edelman et al., in METHODS IN ENZYMOLOGY VOL. 1, page 422 (Academic Press 1967), and Coligan at pages 2.8.1-2.8.10 and 2.10.-2.10.4.

Known Antibodies

Although antibodies against HLA-DR or CD74 are preferred, the poxvirus vaccine can alternatively be made by using an antibody that binds to or is reactive with another antigen on the surface of the target cell. Preferred additional MAbs may comprise a humanized, chimeric or human MAb reactive with CD209 (DC-SIGN), CD34, CD205, TLR 2 (toll-like receptor 2), TLR 4, TLR 7, TLR 9, BDCA-2, BDCA-3 or BDCA-4.

Such antibodies may be obtained from public sources like the American Type Culture Collection or from commercial antibody vendors. For example, antibodies against CD209(DC-SIGN), CD34, BDCA-2, TLR2, TLR 4, TLR 7 and TLR 9 may be purchased from Santa Cruz Biotechnology, Inc. (Santa Cruz, Calif.). Antibodies against CD205 and BDCA-3 may be purchased from Miltenyi Biotec Inc. (Auburn, Calif.). Numerous other commercial sources of antibodies are known to the skilled artisan.

These are exemplary only and a wide variety of other antibodies and their hybridomas are known in the art. The skilled artisan will realize that antibody sequences or antibody-secreting hybridomas against almost any APC-associated antigen may be obtained by a simple search of the ATCC, NCBI and/or USPTO databases for antibodies against a selected target antigen of interest. The antigen binding domains of the cloned antibodies may be amplified, excised, ligated into an expression vector, transfected into an adapted host cell and used for protein production, using standard techniques well known in the art.

Immunoconjugates

In various embodiments, the poxvirus vaccine may be administered as an immunoconjugate. Many methods for making covalent or non-covalent conjugates with antibodies or fusion proteins are known in the art and any such known method may be utilized.

For example, an antigenic peptide can be attached at the hinge region of a reduced antibody component via disulfide bond formation. Alternatively, such agents can be attached using a heterobifunctional cross-linker, such as N-succinyl 3-(2-pyridyldithio)propionate (SPDP). Yu et al., Int. J. Cancer 56:244 (1994). General techniques for such conjugation are well-known in the art. See, for example, Wong, CHEMISTRY OF PROTEIN CONJUGATION AND CROSS-LINKING (CRC Press 1991); Upeslacis et al., “Modification of Antibodies by Chemical Methods,” in MONOCLONAL ANTIBODIES: PRINCIPLES AND APPLICATIONS, Birch et al. (eds.), pages 187-230 (Wiley-Liss, Inc. 1995); Price, “Production and Characterization of Synthetic Peptide-Derived Antibodies,” in MONOCLONAL ANTIBODIES: PRODUCTION, ENGINEERING AND CLINICAL APPLICATION, Ritter et al. (eds.), pages 60-84 (Cambridge University Press 1995).

Dock and Lock (DNL) Method

In alternative embodiments, subunit-based vaccines comprising immunoconjugates may be made by other techniques. One technique for conjugating virtually any protein or peptide to any other protein or peptide is known as the dock-and-lock (DNL) technique. The DNL method exploits specific protein/protein interactions that occur between the regulatory (R) subunits of cAMP-dependent protein kinase (PKA) and the anchoring domain (AD) of A-kinase anchoring proteins (AKAPs) (Baillie et al., FEBS Letters. 2005; 579:3264. Wong and Scott, Nat. Rev. Mol. Cell. Biol. 2004; 5:959).

PKA, which plays a central role in one of the best studied signal transduction pathways triggered by the binding of the second messenger cAMP to the R subunits, was first isolated from rabbit skeletal muscle in 1968 (Walsh et al., J. Biol. Chem. 1968; 243:3763). The structure of the holoenzyme consists of two catalytic subunits held in an inactive form by the R subunits (Taylor, J. Biol. Chem. 1989; 264:8443). Isozymes of PKA are found with two types of R subunits (RI and RII), and each type has α and β isoforms (Scott, Pharmacol. Ther. 1991; 50:123). The R subunits have been isolated only as stable dimers and the dimerization domain has been shown to consist of the first 44 amino-terminal residues (Newlon et al., Nat. Struct. Biol. 1999; 6:222). Binding of cAMP to the R subunits leads to the release of active catalytic subunits for a broad spectrum of serine/threonine kinase activities, which are oriented toward selected substrates through the compartmentalization of PKA via its docking with AKAPs (Scott et al., J. Biol. Chem. 1990; 265; 21561).

Since the first AKAP, microtubule-associated protein-2, was characterized in 1984 (Lohmann et al., Proc. Natl. Acad. Sci. USA. 1984; 81:6723), more than 50 AKAPs that localize to various sub-cellular sites, including plasma membrane, actin cytoskeleton, nucleus, mitochondria, and endoplasmic reticulum, have been identified with diverse structures in species ranging from yeast to humans (Wong and Scott, Nat. Rev. Mol. Cell. Biol. 2004; 5:959). The AD of AKAPs for PKA is an amphipathic helix of 14-18 residues (Carr et al., J. Biol. Chem. 1991; 266:14188). The amino acid sequences of the AD are quite varied among individual AKAPs, with the binding affinities reported for RII dimers ranging from 2 to 90 nM (Alto et al., Proc. Natl. Acad. Sci. USA. 2003; 100:4445). Interestingly, AKAPs will only bind to dimeric R subunits. For human RIIα, the AD binds to a hydrophobic surface formed by the 23 amino-terminal residues (Colledge and Scott, Trends Cell Biol. 1999; 6:216). Thus, the dimerization domain and AKAP binding domain of human Mkt are both located within the same N-terminal 44 amino acid sequence (Newlon et al., Nat. Struct. Biol. 1999; 6:222; Newlon et al., EMBO J. 2001; 20:1651), which is termed the DDD herein.

DDD of Human RIIα and AD of AKAPs as Linker Modules

We have developed a platform technology to utilize the DDD of human RIIα and the AD of a AKAPs as an excellent pair of linker modules for docking any two entities, referred to hereafter as A and B, into a noncovalent complex, which could be further locked into a stably tethered structure through the introduction of cysteine residues into both the DDD and AD at strategic positions to facilitate the formation of disulfide bonds. The general methodology of the “dock-and-lock” approach is as follows. Entity A is constructed by linking a DDD sequence to a precursor of A, resulting in a first component hereafter referred to as a. Because the DDD sequence would effect the spontaneous formation of a dimer, A would thus be composed of a₂. Entity B is constructed by linking an AD sequence to a precursor of B, resulting in a second component hereafter referred to as b. The dimeric motif of DDD contained in a₂ will create a docking site for binding to the AD sequence contained in b, thus facilitating a ready association of a₂ and b to form a binary, trimeric complex composed of a₂b. This binding event is made irreversible with a subsequent reaction to covalently secure the two entities via disulfide bridges, which occurs very efficiently based on the principle of effective local concentration because the initial binding interactions should bring the reactive thiol groups placed onto both the DDD and AD into proximity (Chmura et al., Proc. Natl. Acad. Sci. USA. 2001; 98:8480) to ligate site-specifically.

In certain alternative embodiments, the poxvirus vaccine immunoconjugates are based on a variation of the a₂b structure, in which each heavy chain of an anti-HLA-DR or anti-CD74 antibody or F(ab′)₂ or F(ab)₂ antibody fragment is attached at its C-terminal end to one copy of an AD moiety. Since there are two heavy chains per antibody or fragment, there are two AD moieties per antibody or fragment. A subunit antigenic peptide is attached to a complementary DDD moiety. After dimerization of DDD moieties, each DDD dimer binds to one of the AD moieties attached to the IgG antibody or F(ab′)₂ or F(ab)₂ fragment, resulting in a stoichiometry of four antigenic peptides per IgG or F(ab′)₂ or F(ab)₂ unit. However, the skilled artisan will realize that alternative complexes may be utilized, such as attachment of the antigenic peptide to the AD sequence and attachment of the anti-HLA-DR or anti-CD74 MAb or fragment to the DDD moiety, resulting in a different stoichiometry of effector moieties. For example, by attaching a DDD sequence to the C-terminal end of each heavy chain of an IgG antibody or F(ab′)₂ fragment, and attaching an AD sequence to the antigenic peptide, a DNL complex may be constructed that comprises one antigenic peptide and one antibody or fragment.

By attaching the DDD and AD away from the functional groups of the two precursors, such site-specific ligations are expected to preserve the original activities of the two precursors. This approach is modular in nature and potentially can be applied to link, site-specifically and covalently, a wide range of substances.

In preferred embodiments, the DDD or AD moiety is covalently attached to an antibody or antigenic peptide to form a fusion protein or peptide. A variety of methods are known for making fusion proteins, including nucleic acid synthesis, hybridization and/or amplification to produce a synthetic double-stranded nucleic acid encoding a fusion protein of interest. Such double-stranded nucleic acids may be inserted into expression vectors for fusion protein production by standard molecular biology techniques (see, e.g. Sambrook et al., Molecular Cloning, A laboratory manual, 2^{nd} Ed, Cold Spring Harbor Press, Cold Spring Harbor, N.Y., 1989). In such preferred embodiments, the AD and/or DDD moiety may be attached to either the N-terminal or C-terminal end of a protein or peptide. However, the skilled artisan will realize that the site of attachment of an AD or DDD moiety may vary. For example, although an AD or DDD moiety may be attached to either the N- or C-terminal end of an antibody or antibody fragment while retaining antigen-binding activity, attachment to the C-terminal end positions the AD or DDD moiety farther from the antigen-binding site and appears to result in a stronger binding interaction (e.g., Chang et al., Clin Cancer Res 2007, 13:5586s-91s). Site-specific attachment of a variety of effector moieties may be also performed using techniques known in the art, such as the use of bivalent cross-linking reagents and/or other chemical conjugation techniques.

Methods of Therapeutic Treatment

Formulations

The poxvirus vaccine can be formulated according to known methods to prepare pharmaceutically useful compositions, whereby the poxvirus vaccine is combined in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

The poxvirus vaccine is preferably administered either subcutaneously or nasally. More preferably, the poxvirus vaccine is administered as a single or multiple boluses via subcutaneous injection. Formulations for administration can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

Additional pharmaceutical methods may be employed to control the duration of action of the poxvirus vaccine. Control release preparations can be prepared through the use of polymers to complex or adsorb the poxvirus vaccine. For example, biocompatible polymers include matrices of poly(ethylene-co-vinyl acetate) and matrices of a polyanhydride copolymer of a stearic acid dimer and sebacic acid. Sherwood et al., Bio/Technology 10:1446 (1992). The rate of release from such a matrix depends upon the molecular weight of the poxvirus vaccine, the amount of poxvirus vaccine within the matrix, and the size of dispersed particles. Saltzman et al., Biophys. J. 55:163 (1989); Sherwood et al., supra. Other solid dosage forms are described in Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof.

Generally, the dosage of an administered poxvirus vaccine for humans will vary depending upon such factors as the patient's age, weight, height, sex, general medical condition and previous medical history. It may be desirable to provide the recipient with a dosage of poxvirus vaccine that is in the range of from about 1 mg/kg to 25 mg/kg as a single administration, although a lower or higher dosage also may be administered as circumstances dictate. A dosage of 1-20 mg/kg for a 70 kg patient, for example, is 70-1,400 mg, or 41-824 mg/m² for a 1.7-m patient. The dosage may be repeated as needed for induction of an immune response.

In alternative embodiments, therapeutic peptides may be administered by an inhalational route (e.g., Sievers et al., 2001, Pure Appl. Chem. 73:1299-1303). Supercritical carbon dioxide aerosolization has been used to generate nano or micro-scale particles out of a variety of pharmaceutical agents, including proteins and peptides (Id.) Microbubbles formed by mixing supercritical carbon dioxide with aqueous protein or peptide solutions may be dried at lower temperatures (25 to 65° C.) than alternative methods of pharmaceutical powder formation, retaining the structure and activity of the therapeutic peptide (Id.) In some cases, stabilizing compounds such as trehalose, sucrose, other sugars, buffers or surfactants may be added to the solution to further preserve functional activity. The particles generated are sufficiently small to be administered by inhalation. In still other alternatives, nasal administration of an aqueous solution may be utilized.

Kits

Various embodiments may concern kits containing components suitable for treating or diagnosing diseased tissue in a patient. Exemplary kits may contain at least one or more poxvirus vaccine immunoconjugates as described herein. If the composition containing components for administration is not formulated for delivery via nasal administration or inhalation, a device capable of delivering the kit components through subcutaneous injection may be included. One type of device is a syringe that is used to inject the composition into the body of a subject. In certain embodiments, a therapeutic agent may be provided in the form of a prefilled syringe or autoinjection pen containing a sterile, liquid formulation or lyophilized preparation.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

Expression Vectors

Still other embodiments may concern DNA sequences comprising a nucleic acid encoding a poxvirus vaccine immunoconjugate, or its constituent proteins. Fusion proteins may comprise an anti-HLA-DR antibody attached to a subunit antigenic peptide. Alternatively the encoded fusion proteins may comprise a DDD or AD moiety attached to an antibody or antigenic peptide.

Various embodiments relate to expression vectors comprising the coding DNA sequences. The vectors may contain sequences encoding the light and heavy chain constant regions and the hinge region of a human immunoglobulin to which may be attached chimeric, humanized or human variable region sequences. The vectors may additionally contain promoters that express the encoded protein(s) in a selected host cell, enhancers and signal or leader sequences. Vectors that are particularly useful are pdHL2 or GS. More preferably, the light and heavy chain constant regions and hinge region may be from a human EU myeloma immunoglobulin, where optionally at least one of the amino acid in the allotype positions is changed to that found in a different IgG1 allotype, and wherein optionally amino acid 253 of the heavy chain of EU based on the EU number system may be replaced with alanine. See Edelman et al., Proc. Natl. Acad. Sci. USA 63:78-85 (1969). In other embodiments, an IgG1 sequence may be converted to an IgG4 sequence.

The skilled artisan will realize that methods of genetically engineering expression constructs and insertion into host cells to express engineered proteins are well known in the art and a matter of routine experimentation. Host cells and methods of expression of cloned antibodies or fragments have been described, for example, in U.S. Pat. Nos. 7,531,327; 7,537,930 and 7,608,425, the Examples section of each incorporated herein by reference.

### EXAMPLES

The following examples are provided to illustrate, but not to limit, the claims of the present invention.

### Example 1

### Immune Response to Poxvirus Subunit Antigenic Peptides

### Materials and Methods

Peptide design. 9-mer or 15-mers peptide sequences bearing multiple potential binding sites for both HLA class I and/or HLA class II molecules were derived from poxvirus open reading frames by visual screening for HLA anchor residues at the correct spacing, or by use of web-based methods (e.g., BIMAS or SYFPEITHI [Parker et al., J Immunol 152:163-75, 1994; Rammensee et al., Immunogenetics 50:213-19, 1999]), with selection based on high potential for specific HLA-binding (Table 2). The nucleotide and amino acid sequences of vIL18BP(C12L), A4L (Boulanger et al., J Virol 72:170-79, 1998), A27L (Chung et al., J Virol 72:1577-85, 1998), or D8L (Hsaio et al., J Virol 73:8750-61) VV antigens were retrieved from NIH GenBank, Accession number: AY243312. These peptides are designated by their gene source and a number (e.g., vIL18BP105 or vD8L118).

TABLE 2
Amino acid number and HLA restriction of poxvirus vIL18BP-
derived peptides, A4L229, and TT830.

| | Peptide length | |
|---|---|---|
| Peptide | (amino acids) | Potential HLA binding^{@} |
| TT830* | 15-mer | HLA-A02, (A03, DR04, DR15) |
| vA4L229^{#} | 9-mer | HLA-A02, (A03, A11) |
| vIL18BP008 | 9-mer | HLA-A02, (A03, A11) |
| vIL18BP110 | 9-mer | HLA-A02, (A03, A11) |
| vIL18BP105 | 15-mer | HLA-A02, A03, (A11), A35, |
| vIL18BP102 | 25-mer | DR01, DR04, DR07, DR11, DR15 |
| vA27L003 | 15-mer | HLA-A01, A02, A03, (A11), |
| | | DR01, DR03, DR04, DR07, DR15 |
| vD8L118 | 15-mer | HLA-A01, A02, A03, A11, DR01, |
| vD8L110 | 25-mer | (DR03), DR04, DR07, DR11, |
| | | (DR15) |

*TT830, Tetanus toxoid T-cell helper peptide;
^{#}A4L229, epitope from vaccinia A4L ORF; vIL18BP, poxvirus IL-18 binding protein-derived peptides (vaccinia C12L).
^{@}Binding potential without parentheses ≧15 (www.SYNPEITHI.de); within parentheses, binding probability of 10-14.
15-mers may contain more than one potential HLA class I binding epitope in addition to HLA class II epitopes. Only a limited number of HLA types are shown.

Peptide amino acid sequences are shown below. Peptides were screened for similarities with the human genome, using the NIH Blast server (http://www.ncbi.nlm.nih.gov/blast/). Peptides with homology to the human proteome were discarded. All newly designed peptides were commercially synthesized at 95% purity (Sigma-Genosys, Woodlands, Tex. USA, New England Peptide, Gardner, Mass.).

| vIL18BP118 | |
|---|---|
| CVLTTLNGV | (SEQ ID NO: 13) |
| vIL18BP102 | |
| KFAHYRFTCVLTTLNGVSKKNIVVLK | (SEQ ID NO: 14) |
| vIL18BP105 | |
| HYRFTCVLTTLNGVS | (SEQ ID NO: 15) |
| vIL18BP110 | |
| CVLTTLNGV | (SEQ ID NO: 16) |
| vIL18BP117 | |
| GVSKKNIWL | (SEQ ID NO: 17) |
| vA4L229 (variola virus) | |
| ALKDLMSSV | (SEQ ID NO: 18) |
| TT830 (Clostridium tetani) | |
| QYIKANAKFIGITEL | (SEQ ID NO: 19) |
| vA27L003-027 | |
| GTLFPGDDDLAIPAT | (SEQ ID NO: 20) |
| vA27L003-012 | |
| GTLFPGDDDLAIPATEFFSTKAAKK | (SEQ ID NO: 28) |
| vA27L004-012 | |
| TLFPGDDDL | (SEQ ID NO: 29) |
| vD8L110-134 | |
| HDDGLIIISIFLQVLDHKNVYFQKI | (SEQ ID NO: 21) |
| vD8L118-132 | |
| SIFLQVLDHKNVYFQ | (SEQ ID NO: 22) |
| vD8L116-124 | |
| IISIFLQVL | (SEQ ID NO: 33) |
| vB5R001-025 | |
| MKTISVVTLLCVLPAVVYSTCTVPT | (SEQ ID NO: 30) |
| vB5R004-018 | |
| ISVVTLLCVLPAVVY | (SEQ ID NO: 31) |
| vB5R008-016 | |
| TLLCVLPAV | (SEQ ID NO: 32) |

Donor samples Buffy coats were obtained from the Blood Center of New Jersey (NJBB) (West Orange, N.J. USA). Other PBMC samples were obtained from local donors after approval for use of human blood by the New England Institutional Review Board (Wellesley, Mass. USA), or from Cellular Technology Limited (CTL) (Shaker Hts, Ohio USA). Table 3 summarizes the donor HLA types, age, and vaccine status. Due to limited numbers of cells in each sample, not all samples were included in every assay.

TABLE 3
Summary of blood donor vaccine status, age, and HLA type.

| | Average | |
|---|---|---|
| | age ± SD | |
| Vaccine status | (range) | % (HLA allele) |
| Vaccinated | 43 ± 11 | 14% (A01), 45% (A02), 9% (A03), 0% |
| (N = 22) | (18-66) | (A11), 11% (DR01), 16% (DR04), 9% |
| | | (DR07), 2% (DR11), 9% (DR15) |
| Unvaccinated | 30 ± 12 | 7% (A01), 50% (A02), 0% (A03), |
| (N = 14) | 17-49) | 4% (A11), 4% (DR01), 0% (DR04), 14% |
| | | (DR07), 14%(DR11), 7% (DR15) |

DNA from donor PBMCs was amplified according to HLA-Typing kit (Biotest, Dreieich, Germany) specifications. HLA type was provided for the CTL, Inc., samples. Vaccinated donors were persons who either stated that they had previously received the live smallpox vaccine, or vaccination status was presumed based on age, while unvaccinated donors were persons who stated they had not received a smallpox vaccination or were born after vaccination ceased in the U.S. Due to limited numbers of cells in most samples, not all samples were tested in all assays. When the HLA type of the donors was not determined by the supplier, PBMCs were typed for HLA by SSP-PCR using the Biotest kit (Biotest, Dreieich, Germany).

Peptide screening Transporter associated with antigen-processing protein-1 and -2 (TAP 1 and 2)-deficient human B/T hybridoma cell line, T2 cells (ATCC, Manassas, Va. USA), which expresses surface HLA-A02 exclusively, and which increases its expression when stabilized by peptide in the antigen presentation groove (Nijman et al., Eur J Immunol 23:1215-19, 1993), was incubated with beta-2-microglobulin and peptides at the indicated concentrations. Due to TAP deficiency, peptides are not processed, and so must be of a length that allows binding to HLA-A02 (9-mer). Analysis of HLA was performed using FITC-labeled W6/32 (BD Pharmingen, San Diego, Calif. USA) and a FACSCALIBUR™ flow cytometer (Becton Dickinson, San Jose, Calif. USA). Binding of the peptide epitopes to human PBMCs obtained from donors was detected by incubation of PBMCs at 1×10⁶/mL with biotinylated peptides, followed by addition of avidin-FITC conjugate to fixed cells, and flow cytometry.

T-cell proliferation and phenotype analysis For evaluation, peptides were screened in vitro against PBMCs from smallpox-vaccinated and naïve donors, using a carboxyfluorescein diacetate succinimidyl ester (Invitrogen, Carlsbad, Calif. USA) based cell proliferation assay (Younes et al., J Exp Med 198:1909-22, 2003). For comparison purposes, peptides derived from the immunodominant poxvirus protein, A4L (Boulanger et al., J Virol 72:170-79, 1998), another from Tetanus Toxoid (TT830) (Demotz et al., J Immunol 142:394-402, 1989), or the HIV gag protein (HIVgag) (Kan-Mitchell et al., J Immunol 172:5249-61, 2010), were included. Briefly, 10-50×10⁶ PBMCs were labeled with CFSE (1.5 μM). 2×10⁵ cells (200 μL) were incubated with indicated concentrations of peptides, Staphylococcus aureus enterotoxin (SEA) (10 ng/mL), or phytohemagglutinin (2.5 μg/mL) (PHA, both from Sigma-Aldrich). Cells were stained with antibodies against CD8 or CD3, and for viability (7-AAD) after 5 days. 20,000 events, gated on live CD3+ lymphocytes, were collected by flow cytometry, and analyzed using Flow-Jo software (Mountain View, Calif. USA). Proliferation was evaluated based on the reduction of CFSE fluorescence. The fluorescence index (FI) of proliferating cells was calculated by dividing the number of cells losing CFSE dye in the presence of the stimulating peptide (test) by the number of cells proliferating in the absence of the peptide (control).

For phenotype analysis, PBMCs in GOLGIPLUG™ (Brefeldin A, 1 μg/mL) were incubated with 10 μg/mL of the indicated peptides, medium control (with PBS added in same volume as peptide stock), or SEA or PHA, for 14 h. Cells were then surface- or intracellularly-stained (after permeabilization) with the indicated fluorescently-labeled antibodies (IFN-γ or IL-2). Cells were also stained for CD8, CD45RA to determine prior encounter with antigen, CCR7 (lymph node homing marker) (38), or CD107a (cytolytic capacity marker) (1). The percentages of CD8+ or CD8− effector-memory (T_{EM}) or terminally differentiated T cells (both CD45RA−CCR7−), central-memory T cells (T_{CM}) (CD45RA−CCR7+), and cytokine-driven differentiated T cells (T_{EMRA}) (CD45RA+CCR7−) (12) in peptide-stimulated and control assays were determined. CD8-negative T cells were considered to contain the CD4+ population.

Antibody analysis A modified ELISA-based method (Makabi-Panzu et al., Vaccine 16:1504-10, 1998) was used to assess serum antibody. Briefly, ELISA plate wells were coated with 10 μg/mL of target peptide. After blocking and washing, test sera were added in 2-fold serial dilutions in PBS. Binding of antibody was detected with peroxidase-conjugated anti-human antibody. Plates were developed with o-phenylenediamine dihydrochloride peroxidase substrate (Sigma-Aldrich, St. Louis, Mo. USA) and the optical density of wells was measured at 490 nm with an ELISA reader.

Data analysis The significance of differences observed under the experimental conditions was determined by Student's t-test with Fisher's corrections for multiple comparisons using Statview+SE software (Abacus Concepts, Berkeley, Calif. USA), or Analysis of Variance (ANOVA) (Excel, Microsoft Corp., Redmond, Wash. USA) as indicated. P<0.05 was considered significant.

Results

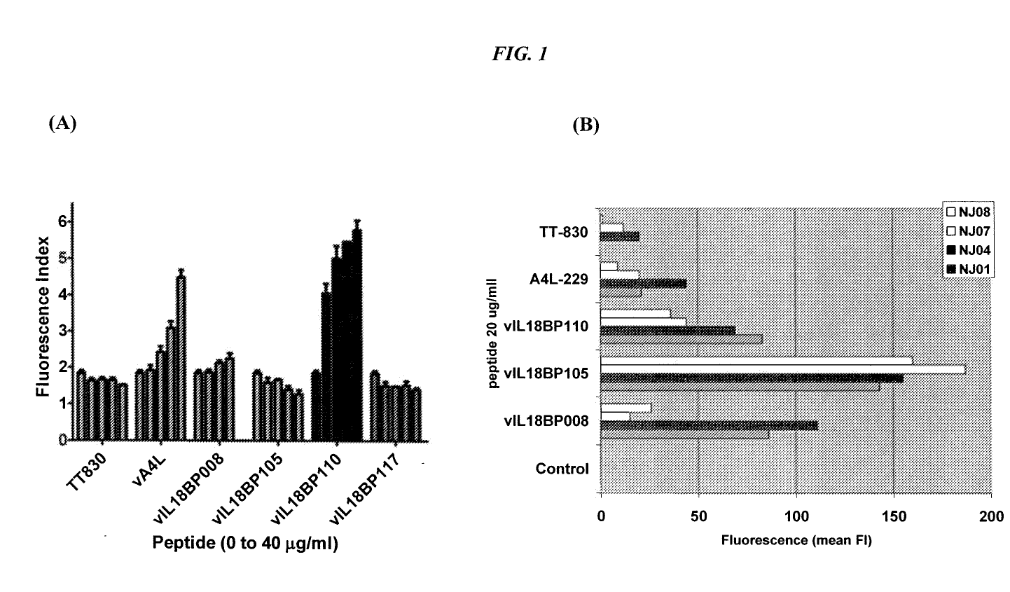

Poxvirus peptide design and screening Poxvirus vIL18BP (SEQ ID NO:23) was parsed into 9-, 15-, or 25-mer peptides based on a high score for HLA-binding potential according to the ranking system of SYFPEITHI or BIMAS, with emphasis on HLA-A02- and HLA-DR04-binding. The vIL18BP-derived peptides were tested for binding to the TAP-deficient T2 hybridoma, which increases expression of HLA-A02 when stabilized by a peptide in the antigen-presenting groove. The 9-mer peptides, vIL18BP110 (SEQ ID NO:16), vIL18BP117(SEQ ID NO:17), and A4L (SEQ ID NO:18) all contain sequences with potential HLA-A0201 binding capability (without processing). Of these peptides, vA4L (SEQ ID NO:18), and vIL18BP110 (SEQ ID NO:16) bound HLA-A02 on T2 cells in a concentration-dependent manner (FIG. 1A). The vIL18BP117 (SEQ ID NO:17) peptide, despite moderate to high probability of binding HLA-A02, did not. Nor did the 15-mer peptides incorporating the sequence of vIL18BP110 (SEQ ID NO:16), which T2 cells cannot process (vIL18BP008, SEQ ID NO:13 and 105, SEQ ID NO:15).

vIL18BP Sequence

(SEQ ID NO: 23)
MRILFLIAFMYGCVHSYVNAVETKCPNLDIVTSSGEFYCSGCVEHMS
KFSYMYWLAKDMKSDEYTKFIEHLGDGIKEDETIRTTDGGITTLRKV
LHVTDTNKFAHYRFTCVLTTLNGVSKKNIWLK

The vIL18BP peptides were also predicted to bind several other HLA haplotypes (Table 2), most of which were represented in the PBMC donor population, summarized in Table 3. When peptides were tested in binding to donor cells, vIL18BP008 (SEQ ID NO:13, 15-mer) and vIL18BP110 (SEQ ID NO:16, 9-mer) demonstrated strong binding to PBMCs from Donors NJ04 (A01/03, DR04) and NJ01 (A11, DR15), and relatively weak binding to NJ07 (A01, DR16) and NJ08 (A01/02,DR16) PBMCs (FIG. 1B). Taking into account donor HLA-types, the predicted HLA target of the peptides, and the T2 results, it can be concluded that vIL18BP110 (SEQ ID NO:16), a 9-mer, does not bind HLA-A01, but binds HLA-A02, -A03, and -A11, all of which were represented by T2 cells, or the donor panel.

The 15-mer, vIL18BP105 (SEQ ID NO:15), was predicted to bind HLA class II DR04 and DR15 (NJ01 and NJ04), and all of class I HLA types represented by the donors except HLA-A01. In addition to HLA-A01, donor NJ08 is HLA-A02-positive, thus HLA-A02 may account for the measured binding. Evidence for binding of vIL18BP105 (SEQ ID NO:15) to HLA-DR16 is suggested by the strong signal from Donor NJ07, which expresses HLA-DR16 and non-binding HLA-A01. While the consensus motif for HLA-DR16 has not been well-characterized (Onion et al., J Gen Virol 88:2417-25, 2007), there is at least one report that suggests the binding motifs of HLA-DR15 and -DR16 share similarities (Zeng et al., J Virol 70:3108-17, 1996).

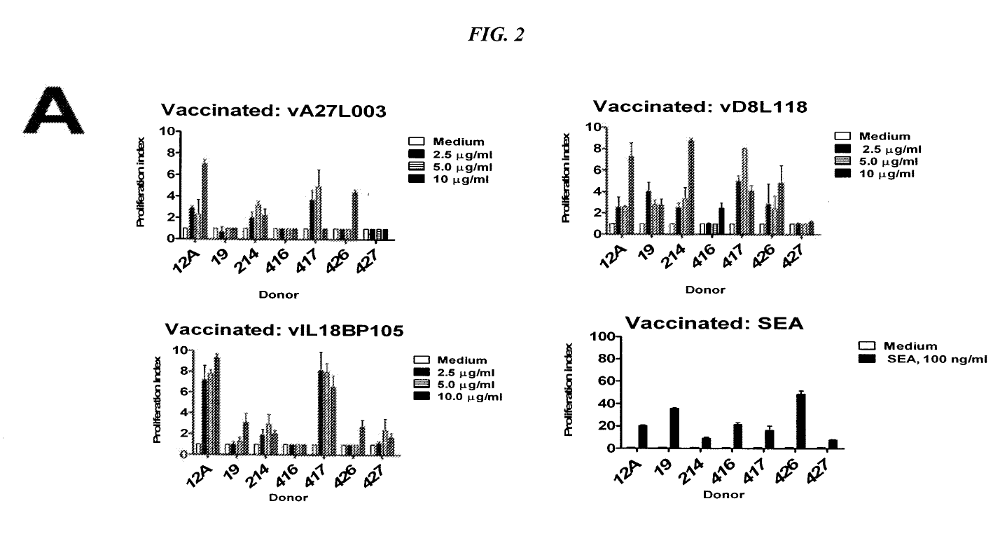

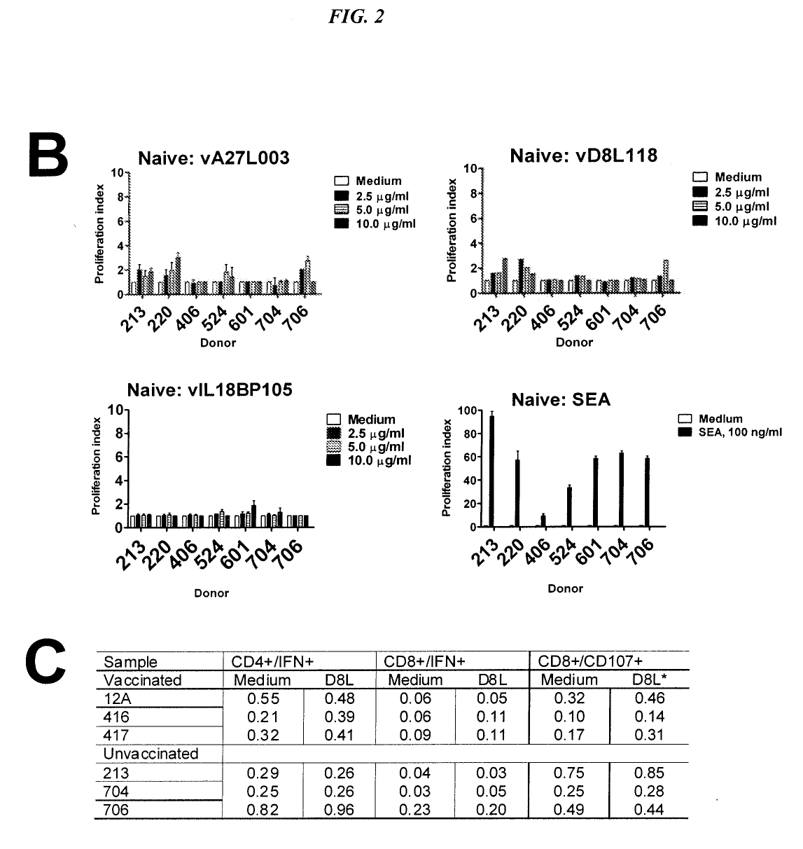

Immunoreactivity of peptides as antigen mimics for T cells was assessed by 5-day CFSE-based proliferation assays, where CFSE-loaded PBMCs from vaccinated or unvaccinated donors were incubated with vIL18BP105 (SEQ ID NO:15) and peptides from two other poxvirus genes, vD8L118 (SEQ ID NO:22) and vA27L003 (SEQ ID NO:20). The results for all the vIL18BP105 (SEQ ID NO:15), assays are summarized in Table 4. Results for concurrent assays for vIL18BP105 (SEQ ID NO:15), vD8L118 (SEQ ID NO:22), and vA27L003 (SEQ ID NO:20) are shown in FIG. 2A (vaccinated donors) and FIG. 2B (unvaccinated donors).

Overall, vIL18BP105 (SEQ ID NO:15), induced significant proliferation of PBMCs from vaccinated donors (Table 4) at a concentration of 10 μg/mL. Vaccinated donor cells also proliferated when incubated with vD8L118 (SEQ ID NO:22) (6 of 7) and vA27L003 (SEQ ID NO:20) (4 of 7, FIG. 2A). These results indicate that vIL18BP105 (SEQ ID NO:15), vD8L118 (SEQ ID NO:22), and vA27L003 (SEQ ID NO:20) include epitopes that are recognized by lymphocytes from smallpox-vaccinated donors. Cells from unvaccinated donors were overall unresponsive to the poxvirus peptides (FIG. 2B). When samples from vaccinated donors (12A, 416, 417) and unvaccinated donors (213, 704, 706) were assayed for markers of activation and intracellular cytokine production in separate experiments (14-h assays), an IFN-γ response was noted in both CD4+ and CD8+ cells. CD8+ cells also expressed the cytolytic capacity marker, CD107a (P<0.05 vs. medium controls, FIG. 2C).

TABLE 4
Summary table of PBMC proliferative responses to vIL18BP105

| | |
|---|---|
| Donor | Fluorescence index (FI) |

| vaccine status | vIL18BP105 | PHA or SEA |
|---|---|---|
| Yes (N = 11) | 5.07 ± 3.37 | 25.27 ± 14.15 |
| No (N = 10) | 1.00 ± 0.46 | 49.68 ± 24.39 |

Concentrations of 10 μg/mL of vIL18BP105 peptide, 2.5 μg/mL of PHA (P), or 10 μg/mL SEA (S) were used. Fluorescence Index (FI) is estimated by dividing the number of cells proliferating in the presence of peptide (or P or S) by the number of cells proliferating in the absence of peptide (or P or S) using the CFSE-based cell proliferation assay described in Materials and Methods. N=number of individual donors. All samples were assayed in triplicate (mean±SD). P<0.001, vaccinated vs. unvaccinated for vIL18BP105 (ANOVA).

Phenotype of proliferating cells To determine the CD4 or CD8 phenotype of the proliferating cells, CFSE-loaded PBMCs from vaccinated and unvaccinated controls incubated with either vA27L003 (SEQ ID NO:20) or vIL18BP105 (SEQ ID NO:15), (5 days) were probed for CD4 or CD8 expression. Both CD4+ (4/5) and CD8+ (2/5) cells proliferated in samples from vaccinated donors, with little to no proliferation of either subset of cells in the unvaccinated donor samples (0 of 3, Table 5).

Further determinations of responding cells' phenotype were performed in 14-hour intracellular cytokine staining assays. Increased IFN-γ production in the CD8+ T cell population was found in samples incubated with vD8L118 (SEQ ID NO:22) (2/5) or vIL18BP105 (SEQ ID NO:15) (2/5) (Table 6, P<0.05). Despite stimulating proliferation in the 5-day CFSE-based assay, vA27L003 (SEQ ID NO:20) did not stimulate IFN-γ or IL-2 increases (not shown). IFN-γ production did not significantly increase in the CD4+ T cells, but isolated samples responded. However, IL-2 production increased significantly in the CD4+ population (vD8L118, SEQ ID NO:22) and in CD8+ cells (vIL18BP105, SEQ ID NO:15) (Table 6; P<0.04).

TABLE 5
CD4+ or CD8+ phenotype of proliferating T cells incubated with vA27L003 or
vIL18BP105 peptides (5-day assay).

| | CD4 | | | | CD8 | | | |
|---|---|---|---|---|---|---|---|---|
| | Cont | PHA | vA27L | vIL18BP | Cont | PHA | vA27L | vIL18BP |
| UNVACCINATED | | | | | | | | |
| NJ01 | 7.73 | 37.44 | Nd | 1.10 | 1.31 | 20.38 | nd | 0.94 |
| E | 0.61 | 11.46 | 1.11 | nd | 0.15 | 5.91 | 0.15 | nd |
| G | 0.92 | 2.40 | 0.63 | nd | 0.21 | 0.46 | 0.00 | nd |
| VACCINATED | | | | | | | | |
| NJ04 | 1.66 | 12.57 | Nd | 6.02 | 1.01 | 6.91 | nd | 3.64 |
| D | 3.52 | 20.84 | 3.52 | nd | 1.65 | 29.22 | 1.22 | nd |
| F | 1.08 | 17.67 | 8.30 | nd | 1.12 | 19.80 | 7.85 | nd |
| H | 4.46 | 49.20 | Nd | 25.73 | 2.75 | 11.00 | nd | 15.14 |
| NJ08 | 3.10 | 26.47 | Nd | 6.25 | 3.76 | 36.29 | nd | 7.26 |

Stored human PBMCs were thawed, incubated with CFSE for 24 h, followed by incubation with either PHA-P (2.5 μg/mL) or 20 μg/mL peptide as indicated, after which they were stained for CD4 or CD8 expression. Analysis was by flow cytometry. Column headings: Cont: medium control; vA27L: vA27L003; vIL18BP: vIL18BP105. Values in bold-face are ≧1.5-fold vs. control.

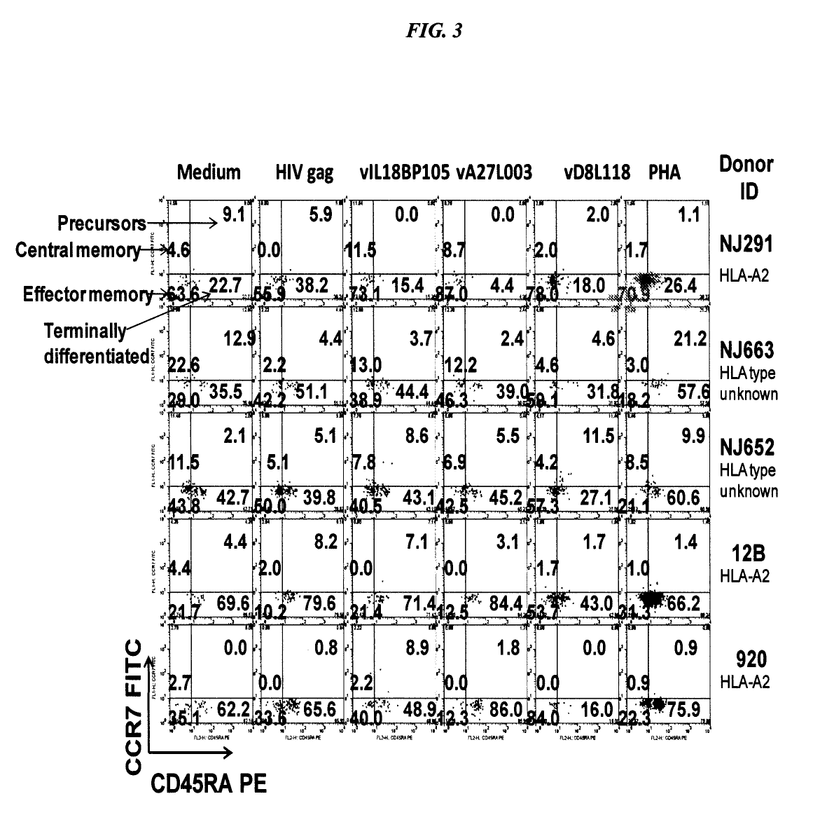

CD8+/IFN-γ-producing T cells from the same vaccinated donors were further analyzed for markers related to memory phenotype by staining for CD45RA, a marker of naïve and a subset of effector CD8 cells (T_{EMRA}), and CCR7, a lymph node homing marker. This analysis differentiates between T_{CM} (CCR7+CD45RA−), precursors (CCR7+CD45RA+), T_{EMRA} (CCR7−CD45RA+), and T_{EM} and terminally differentiated (CCR7−CD45RA−) cell populations. The cell types that developed were CCR7−CD45RA− (T_{EM} or terminally-differentiated effector) (FIG. 3). The vD8L118 (SEQ ID NO:22) antigen peptide was most active in generating these cell types (P<0.019 vs. medium controls). In addition, 2 donors in each assay also responded similarly to vIL18BP105 (SEQ ID NO:15), and vA27L003 (SEQ ID NO:20).

TABLE 6
IL-2 or IFN-γ production by CD4 and CD8 T cells incubated with
poxvirus peptides for 14 h.

| | Medium | | | | | | | |
|---|---|---|---|---|---|---|---|---|
| | control | | PHA | | vD8L118 | | vIL18BP105 | |
| IFN-γ | CD4 | CD8 | CD4 | CD8 | CD4 | CD8 | CD4 | CD8 |
| Donor: | | | | | | * | | * |
| NJ291 | 0.06 | 0.01 | 1.25 | 0.48 | 0.16 | 0.08 | 0.04 | 0.02 |
| NJ663 | 0.49 | 0.06 | 4.68 | 0.31 | 0.40 | 0.10 | 0.60 | 0.10 |
| NJ652 | 1.17 | 0.2 | 8.05 | 0.62 | 0.97 | 0.37 | 1.34 | 0.25 |
| 12B | 0.3 | 0.01 | 5.66 | 2.28 | 0.45 | 0.11 | 0.48 | 0.02 |
| 920 | 0.37 | 0.02 | 7.05 | 0.49 | 0.13 | 0.02 | 0.41 | 0.02 |
| IL-2 | | | | | * | | | * |
| NJ291 | 0.02 | 0.01 | 0.26 | 0.02 | 0.21 | 0.01 | 0.02 | 0.04 |
| NJ663 | 0.22 | 0.01 | 1.97 | 0.11 | 2.93 | 0.11 | 0.17 | 0.05 |
| NJ652 | 0.14 | 0.03 | 0.60 | 0.15 | 2.44 | 0.27 | 0.20 | 0.08 |
| 12B | 0.03 | 0 | 0.30 | 0.03 | 0.08 | 0.01 | 0.06 | 0.01 |
| 920 | 0.06 | 0.01 | 0.71 | 0.06 | 1.20 | 0.04 | 0.08 | 0.02 |

Values shown are percent positive cells for each cytokine for each donor after 14 h of incubation with the designated peptide. Cells were stained for CD8, and the CD8-negative lymphocytes were considered CD4+. CD8+ cells consisted of 10-30% of total lymphocytes. All donors were vaccinated against smallpox. Concentration of peptides: 10 μg/mL; PHA, 2.5 μg/mL. Values in bold-face are ≧1.5-fold above medium control. Response to HIV peptide and vA27L003 was not significantly different than medium-only controls. *P<0.05 for CD8/IFN-γ/vD8L118 and vIL18BP105 vs. medium control: P<0.04 for CD4/IL-2/vD81118 vs. medium control; P<0.013 for CD4/IL-2/vIL18BP105 vs. medium control (t-test).

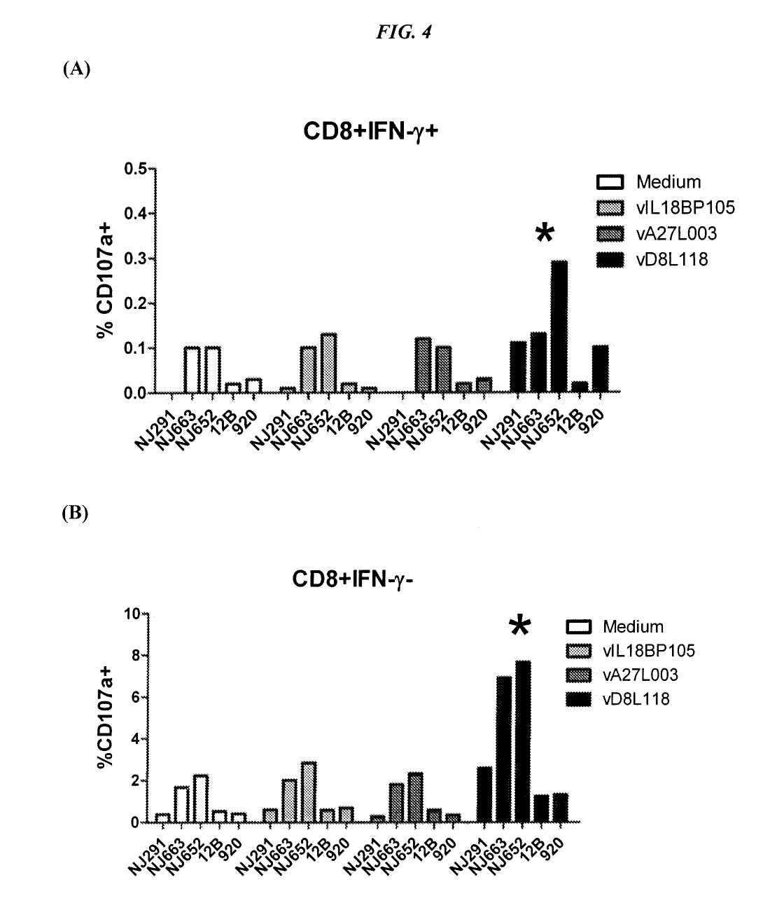

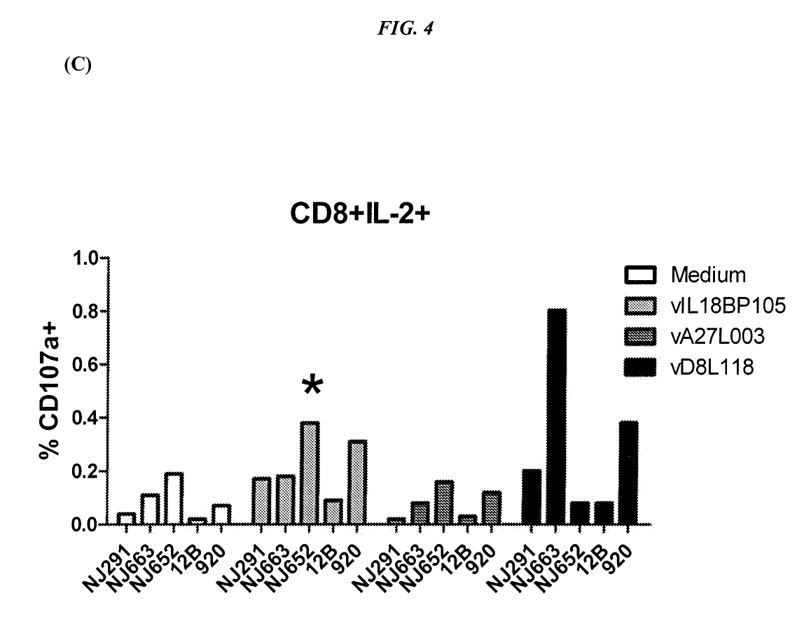

The capacity of the CD8+ effector cell population to degranulate, i.e., their ability to perform effector function, was assayed by determination of the expression of CD107a (Berhanu et al., J Virol 82:3517-29, 2008) (FIG. 4). In both the CD8+IFN-γ+ and the CD8+IFN-γ-populations, CD107a expression increased 2-7-fold in 3 of 5 PBMC samples incubated with vD8L118 (SEQ ID NO:22) (P<0.04). Increased CD107a was also measured in the CD8+IL-2+ population when incubated with vIL18BP105 (SEQ ID NO:15), (P<0.01) and vD8L118 (SEQ ID NO:22), although the latter did not achieve significance.

CD8+IFN-γ+ cells from unvaccinated donors were unresponsive to the peptides in similar 14-hour intracellular cytokine staining assays (not shown).

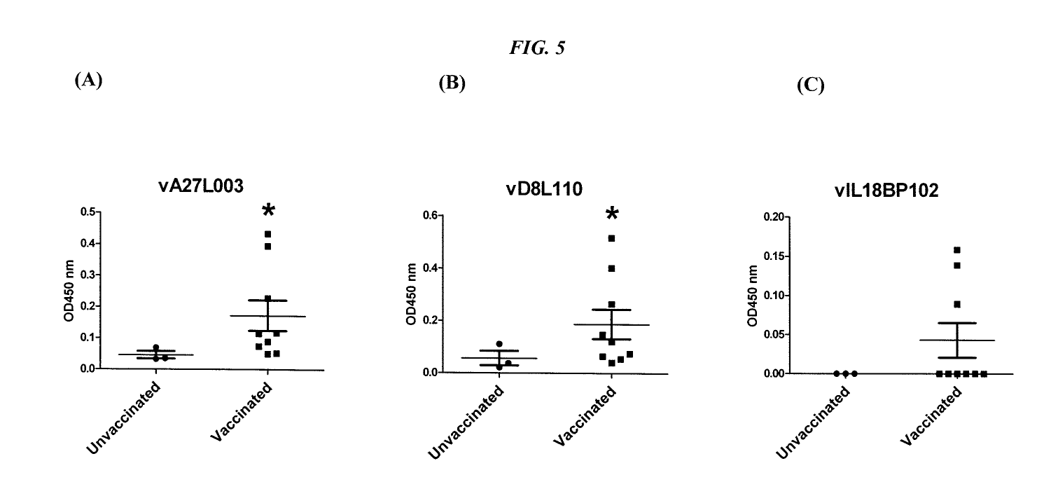

Serum antibody titers Antibody against poxvirus is required for protection upon secondary exposure, and the presence of anti-vaccinia antibody is maintained in 90% of vaccinees for decades after vaccination (Hammarlund et al., Nat Med 9:1131-37, 2003). Therefore, serum antibody from previously vaccinated patients would be directed toward immunologically relevant B-cell epitopes. To determine if the antigen peptides' sequence included recognizable B-cell epitopes, 1:200 diluted sera from vaccinated and unvaccinated donors were tested with the peptides vA27L003 (SEQ ID NO:20) (15-mer), vIL18BP102, and vD8L110 (25-mers). The results (FIG. 5) show that serum antibody to the vD8L110 and vA27L003 (SEQ ID NO:20) peptides was higher overall, and significantly above that from unvaccinated individuals (P<0.05). Although 3 donors produced antibody that recognized vIL18BP102, the overall results did not achieve significance. The results suggest that the experimental peptides contained one or several sequences that are B-cell epitopes. The presence of anti-peptide antibodies did not differ according to age of donor or time since vaccination (not shown).

Discussion

Inclusion of antigenic peptides in an alternative poxvirus vaccine requires that they be relevant targets of human immunity. The results described above determined whether or not specific peptides derived from poxvirus antigens were able to elicit memory responses in PBMCs from vaccinated donors. The epitopes were derived from three poxvirus antigens, including an antigen (vIL18BP) that is uncharacterized in host immunity, as well as the known poxyiral envelope antigens, A27L and D8L, which are characterized for host protection, but for which specific epitopes are not characterized (Chung et al., J Virol 72:1577-85, 1998; Hsaio et al., J Virol 73:8750-61, 1999). The types of responses elicited by each peptide varied.

Poxvirus IL18BP modulates host innate immunity by neutralizing NK cell IL-18 which, in turn, prevents IFN-γ production. Recognition of one set of peptides from vIL18BP, vIL18BP105 (SEQ ID NO:15), and its derivatives, by CD4+ and CD8+ cells, and serum antibody from vaccinated donors, confirms the hypothesis that this, and most likely other, transiently-expressed viral host-response modulators are targets of host immunity. Neutralization of vIL18BP may aid in protection from initial infection, and therefore, establishment of infection, as was demonstrated recently for poxvirus type I IFN-binding protein (Xu et al., J Exp Med 205:981-92, 2008).

The design of an antigen epitope-based vaccine strategy requires that the viral components interact with HLA for T-cell development. The peptides in this study were predicted to bind several defined HLA haplotypes. But, despite predictions of HLA-binding, only some epitopes bound, as was demonstrated by the peptides from the C12L sequence (vIL18BP).

Peptides from C12L (vIL18BP), as well as the antigens A27L and D8L, elicited proliferation by CD4+ and CD8+ cells from vaccinated donors, indicating that APCs take up the peptides and process them for presentation in the context of both HLA class I for CD8+, and class II for CD4+ lymphocytes.

Immunity to poxvirus is dependent on both cellular and humoral immunity (Dunne et al., Blood 100:933-40, 2002; Ferrier-Rembert et al., Viral Immunol 20:214-20, 2007; Meseda et al., Clin Vaccine Immunol 16:1261-71, 2009; Xu et al., J Immunol 172:6265-71, 2004), both of which require T-cell help for class switching and affinity maturation. The proximity of T and B epitopes within a polypeptide may impact antibody production due to differences in their presentation. However, natural B-cell epitopes are quite often proximal to HLA-binding regions (Simitsek et al., J Exp Med 181:1957-63, 1995). A number of factors may influence the formation of antibody, including HLA type (Quaratino et al., J Immunol 174:557-63, 2005).

In the studies described above, we demonstrated measurable antibody in a sub-set of ⅓ of the vaccinated donors. In addition, the peptides encompassed linear sequences, and therefore the antibody recognized linear epitopes, which can include as few as 3 amino acids (Tahtinen et al., Virology 187:156-64, 1992). In the case of vIL18BP105 (SEQ ID NO:15), further evidence of the presence of a relevant B-cell epitope is provided by mouse studies, where serum antibody against vIL18BP105 (SEQ ID NO:15) recognized full length recombinant vIL18BP(C12L) protein (not shown). Overall, these results show that the antigen peptides used in this study present T- and B-cell targets of human response.

Cytokine and marker production revealed that vIL18BP105 (SEQ ID NO:15) and vD8L118 (SEQ ID NO:22) elicited IL-2 production, which preserves the proliferation capacity of T cells, even in the absence of CD4 help (Zimmerli et al., Proc Natl Acad Sci USA 102:7239-44, 2005). This supports the proliferation data and further demonstrates the utility of vIL18BP peptides for subunit-based vaccines. IFN-γ production by peptide-stimulated CD8+T_{EM} cells has multiple effects, including induction of anti-viral effector function. Thus, when vD8L118 (SEQ ID NO:22) stimulated CD8+T cells with effector and proliferative potential, the results were similar to that reported for cells incubated with virus (Laouar et al., Plos One 3:e4089, 2008). IFN-γ production also characterizes generation of a Th1 response, which is necessary for development of cytotoxicity against vaccinia (Meseda et al., Clin Vaccine Immunol 16:1261-71, 2009; Xu et al., J Immunol 172:6265-71, 2004). Additionally, production of IL-2 and IFN-γ by CD4+ cells implicates helper Th1-oriented T-cell participation. The A27L peptide did not stimulate increased IFN-γ or IL-2 in CD8+ or CD4+ cells, even though T cells proliferated when incubated with this peptide. The lack of activity in the A27L samples may be due to different kinetics of response, or stimulation of alternate populations of cells which produce different cytokines or interleukins, such as IL-4, for which we did not assay.

Our results with respect to CD107a, a marker of cytolytic capacity, are similar to a recent study, where a subset of CD4+ cells from revaccinated donors expressed CD107a upon stimulation by vaccinia virus (Puissant-Lubrano et al., J Clin Invest 120:1636-44, 2010). In our study, CD8+ cells from vaccinated donors demonstrated enhanced expression of this marker upon stimulation by the peptides.

In contrast to the finding of Combadiere et al. (J Exp Med 199:1585-89, 2004), we did not observe loss of ability to produce cytokines in response to vaccinia antigens when smallpox vaccination took place more than 45 years previously.

In summary, we have presented evidence that subunit antigenic peptides from 3 poxvirus antigens are capable of stimulating recall responses from vaccinated donors, including T-cell proliferation, expression of cytokines, and serum antibody recognition of B-cell epitopes. One antigenic epitope was from a heretofore uncharacterized host defense modulator produced by vaccinia, the IL18BP. The results presented here show that development of an alternative vaccine against poxvirus using select peptide epitopes could produce immunity without the hazards of vaccination with active virus. An advantage of this virus-free approach over immunization with attenuated forms of poxvirus, the virulence genes of which are often deleted or mutated, is that the immunologically-relevant portions of any poxvirus gene, as well as altered genes, can be included.

### Example 2

### Conjugation of APC-Targeting Antibody to Subunit Antigenic Peptides for Poxvirus Vaccines

Summary

The vIL18BP105 (SEQ ID NO:15) was conjugated to the anti-HLA-DR antibody, L243, for better presentation to the immune system, and used to immunize HLA-DR04-expressing transgenic (tg) mice. Conjugated vIL18BP105 (CIL18BP105) was more readily taken up by human and HLA-DR transgenic mouse cells than free vIL18BP105 (SEQ ID NO:15). Splenocytes from HLA-DR04 transgenic mice immunized with CIL18BP105 proliferated in vitro when stimulated with vIL18BP105 (SEQ ID NO:15). Proliferation of CIL18BP105-inoculated mouse splenocytes involved CD3+CD4+CD45RA− cells. Proliferation was accompanied by interferon-γ production (quantitative sandwich ELISA). CIL18BP105-innoculated mice also showed early and rapidly rising titers of peptide-specific antibodies, 4 times that of vIL18BP105-injected controls at day 7 after the first boost. At a later time, both CIL18BP105 and vIL18BP105 (SEQ ID NO:15) induced IgG2a and IgG1, suggesting the initiation of both Th1 and Th2 immunity. Serum antibody from CIL18BP105-immunized mice recognized whole recombinant C12L protein. These results demonstrate that conjugation of antigenic peptides to anti-HLA-DR antibody boosts immunogenicity and enhances peptide delivery to antigen-presenting cells expressing HLA-DR.

Methods

HLA-DR antibody-conjugates Peptides that were found to stimulate proliferation of immune donor PBMCs were conjugated with L243 antibody using the heterobifunctional cross-linker, sulfosuccinimidyl-4-(N-maleimidomethyl)cyclohexane-1-carboxylate (Sulfo-SMCC), “SMCC”, containing H-hydroxysuccinimide (NHS) ester and maleimide groups, following the manufacturer's protocol (Pierce, Rockford, Ill., USA). SMCC interacts with primary amine of the antibody through its NHS ester groups to form amide bonds, and the maleimide groups form thioester bonds with the free sulfhydryl group of a C-terminal cysteine on the peptide. Briefly, 1 ml of a 1 mg/ml solution of antibody in PBS was reacted with 20 μL of a 1 mg/ml solution of freshly prepared SMCC in PBS for 2 hours at 4° C. Following incubation; excess SMCC was removed through a PBS pre-equilibrated desalting column. The activated antibody was collected and then incubated with the peptide (which bore a C-terminal cysteine) for another 2 hours (or overnight) at 4° C. The conjugate was purified by size exclusion using a P60 fine cross-linked bead column (BioRad, Hercules, Calif., USA) to remove free peptide. Peptide conjugation efficiency was assessed by SDS-PAGE using a 5%-20% gradient gel. Before being injected into mice, conjugate preparations were filter-sterilized through a 0.22-gm PVDF filter (Millipore, Bedford, Mass.), and emulsified in incomplete Freund's adjuvant (IFA).

Immunization of mice Six-to-eight week old female C57BL/6J (B6) transgenic (tg) mice expressing HLA-DR04 (HLA-DR tg) were obtained from Taconic (Germantown, N.Y., USA). Mice were maintained in a pathogen-free area of our facility. For immunizations, groups of 3 mice were primed, and then boosted twice at two-week intervals by the subcutaneous route, with 25 μg of vIL18BP105 (SEQ ID NO:15) peptide emulsified in IFA in either free, or antibody-conjugated, form. Mice injected with IFA-emulsified PBS served as naive controls. Blood was collected at one-week intervals from priming to sacrifice, which was 7 days after the final boost. Spleen samples were collected at sacrifice. Serum for antibody detection and isotyping by ELISA was prepared from blood after overnight coagulation at 4° C. Splenocytes used in CFSE-based T-cell proliferation assays and TCR repertoire analysis, were isolated by mechanical disruption of spleens through stainless steel mesh.

Antibody production analysis and isotyping determination A modified ELISA-based method from a previous report was used (Makabi-Panzu et al, 1998) to assess antibody production and isotype. Briefly, ELISA plate wells were coated with 10 μg/ml of peptide in PBS and incubated overnight at 4° C. They were then blocked with skim milk/PBS for 30 minutes at 37° C. and washed with PBS containing 0.05% Tween 20 (PBST). Test sera were either added in 2-fold serial dilution for antibody titer, or as a 1:200 dilution for isotype determination. Plates were incubated with sera for 2 h at room temperature. Excess serum was removed by washing three times with PBST, and peroxidase-conjugated goat anti-mouse (or peroxidase-conjugated sheep anti-mouse IgA, IgG1, IgG2a, IgG2b, IgG3 and IgM in case of isotyping) at 1:1000 dilution was added for 45 min at room temperature. Following this incubation, wells were washed, peroxidase substrate was added, and after development, the OD of wells was measured at 490 nm with an ELISA reader.

T-cell proliferation assay and TCRVβ repertoire analysis T-cell proliferation for either donor PBMCs or murine splenocytes was assessed using a 5-day CFSE-based cell proliferation assay as reported previously (Younes et al, 2003). Briefly, 10-50×10⁶ PBMC or splenocytes were labeled with CFSE at a final concentration of 1.5 μM. Cells were washed twice in PBS and re-suspended in complete RPMI medium at 10⁶ cells/ml. 2×10⁵ cells were incubated with indicated concentrations of peptides or PHA (2.5 μg/ml) for positive control wells. Cells were stained with CD4-APC, CD8-PE with 7-AAD or CD3-perCp after 5 days of in vitro incubation at 37° C. in a 5% CO₂ atmosphere. A minimum of 20,000 events gated on live CD3+ lymphocytes were collected on a FACScalibur flow cytometer, and analyzed using Flow Jo software. T-cell proliferation was evaluated based on the reduction of CFSE fluorescence of growing cells. An integrated cell proliferation Flow Jo program was used for analysis. The fluorescence index of proliferating cells was calculated by dividing the number of cells losing the CFSE dye in the presence of the stimulating peptide (test) by the number of cells proliferating in the absence of the peptide (control).

For the TCRVβ repertoire analysis, washed splenocytes from immunized or naive mice were washed again with complete RPMI-1640 medium and with staining buffer, then pre-stained for T-cell surface markers as described above, for 20 min at 4° C., before being incubated again for 15 min at 4° C. with the blocking 2.4G2 anti-FcRIII/I mAb. The cells were then stained with an appropriate fluorescently labeled anti-TCRVβ antibody without removal of the FcR-blocking mAb. Following this last incubation, the cells were washed with stain buffer and analyzed by flow cytometry.

Data analysis The significance of differences observed under the experimental conditions was determined using one way analysis of variance followed as appropriate by a t-test with Fisher's corrections for multiple comparisons using Statview+SE software (Abacus Concepts, Berkeley, Calif.). P<0.05 was considered significant.

Results

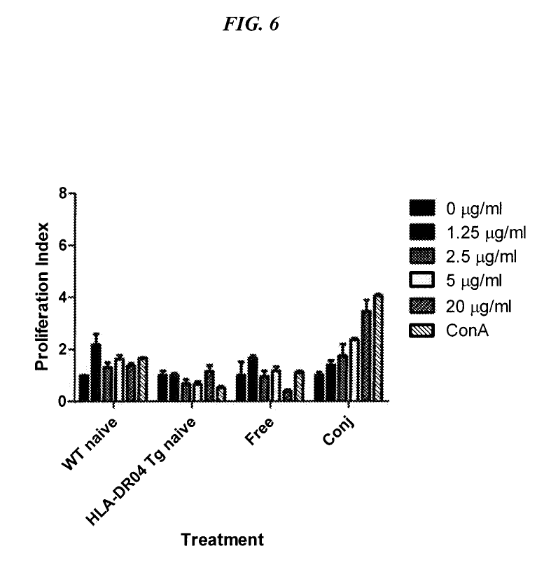

In vitro T-cell proliferation in response to vIL18BP105 (SEQ ID NO:15) peptide To test whether conjugation of a sub-unit antigen to an APC-targeting mAb would generate an enhanced immune response, the peptide vIL18BP105 (SEQ ID NO:15) was conjugated chemically to the mAb L243 (CIL18BP105) and used to immunize mice. The results were compared to mice given PBS/IFA (naive) and free vIL18BP105 (SEQ ID NO:15) in IFA. Using a 5-day CFSE-based in vitro cell proliferation assay, splenocytes from CIL18BP105-immunized mice proliferated in a concentration-dependent manner. Cells from naive or free-peptide immunized mice were relatively unresponsive (FIG. 6). The TCRVf3 repertoire of CD4-positive splenocytes from HLA-DR04 tg mice following immunization with either form of vIL18BP105 (SEQ ID NO:15) skewed toward TCRVβ8.3 (not shown).

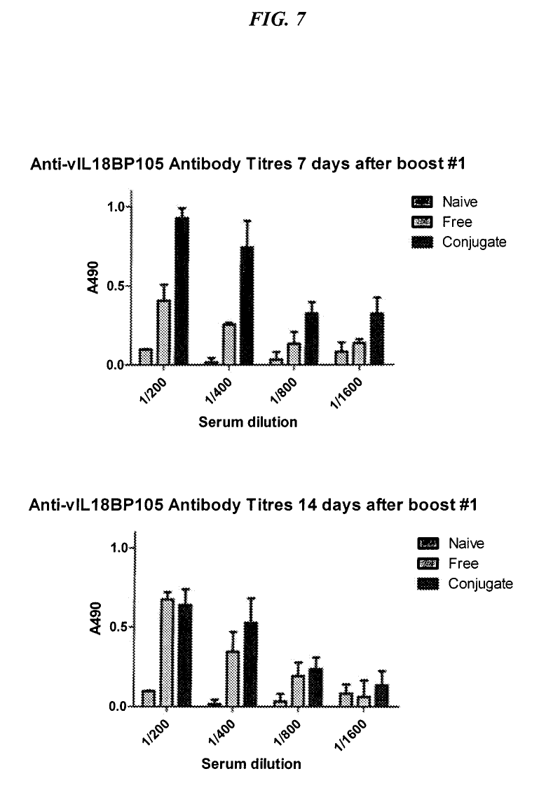

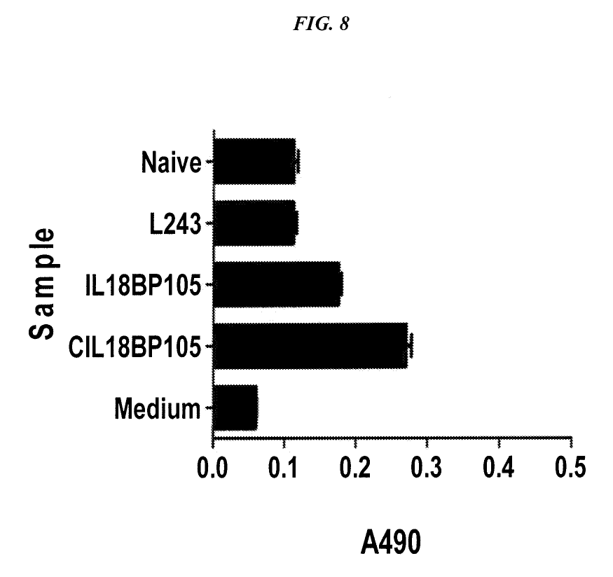

Antibody production in response to vIL18BP105 (SEQ ID NO:15) peptide Humoral immunity to poxvirus is essential for protection against infection. Therefore, the antibody response against CIL18BP105 versus vIL18BP105 (SEQ ID NO:15) was investigated in the immunized HLA-DR04 tg mice. The results are shown in FIG. 7. At day 7 after the first boost (day 21 after priming), CIL18BP105-injected mice displayed higher peptide-specific antibody production than mice injected with vIL18BP105 (SEQ ID NO:15). But, at 14 days after the first boost, the amounts of antibody were similar. Both IgG1 and IgG2a isotypes were induced by CIL18BP105 and vIL18BP105 (SEQ ID NO:15), but CIL18BP105 caused more production of IgG1 antibodies than its free counterpart (not shown). The production of IgG1 and IgG2a suggests a mature antibody response with T-cell help. Both Th1 and Th2 helper cell participation is also suggested by this antibody response. Serum antibody from CIL18BP105-immunized mice reacted strongly with whole vIL18BP protein (C12L) (FIG. 8), indicating that subunit antigenic peptide conjugated to anti-APC antibody is capable of inducing a systemic immune response against intact virions. Immunization with CIL18BP105 was more effective than immunization with vIL18BP105 (SEQ ID NO:15) at promoting interferon-γ production from splenocytes stimulated in vitro with vIL18BP105 (SEQ ID NO:15) peptide (Table 7).

These results show that the poxvirus sub-unit peptide, vIL18BP105 (SEQ ID NO:15), induced both cellular and humoral immune responses in HLA-DR04 tg mice when conjugated with the anti-HLA-DR antibody, L243. T-cell proliferative responses, which are indicative of cell-mediated immunity, were especially enhanced by the antigen-L243 conjugate. Antibody production rose more quickly in the CIL18BP105-immunized mice. The peptide-antibody conjugate induced higher titers of antibody earlier than the free peptide. These results show that T-cell response against a relatively small peptide antigen can be elicited successfully by conjugation to L243.

TABLE 7
Interferon-γ Production From Immunized Mice

| | #CIL18BP105- | IL18BP105- | Naive- |
|---|---|---|---|
| Treatment | splenocyte | splenocyte | splenocyte |
| Medium | 00.00 + 00.00 | 00.00 + 00.00 | 00.00 + 00.00 |
| vIL18BP105, | 101.03 + 11.61* | 25.78 + 11.27 | 38.32 + 11.27 |
| 10 ug/ml | | | |
| Con A, 5 ug/ml | 373.15 + 4.23 | 84.68 + 10.00 | 149.00 + 12.46 |
| SEA, 100 | 363.00 + 21.44 | 148.24 + 00.00 | 66.46 + 17.55 |
| ng/ml | | | |

Conclusions

A vaccine against poxvirus requires Th1 and Th2 immune responses, cell-mediated and humoral immunity, and a suitable pool of memory CD4 T cells (Belyakoc et al, Proc. Natl. Acad. Sci. USA 100: 9458-9463, 2003). The results presented show that sub-unit antigens conjugated to APC-targeting antibody can enhance and to induce Th1, Th2, and humoral immune responses.

### Example 3

### Nasal Administration of Subunit Vaccine

Mice (HLA-DR04 Tg) are anesthetized and vaccine is administered (15-25 μg peptide total) intranasally (i.n.) (10 μl/nostril). Vaccine is either free peptide, or peptide-L243 conjugate. For these experiments, the adjuvant is the calcium phosphate adjuvant described by He et al. (Clin Diagnos Lab Immunol 9:1021-1024, 2002) (10 μg/dose of antigen). Controls consist of unimmunized (naive) mice, mice immunized with the whole viral protein (i.n.), systemically immunized mice (peptide, sub-cutaneous (s.c.)), and mice immunized with carrier/adjuvant only (i.n.). Equal amounts of peptide are administered in each case. Mice are boosted twice using the same route as prime, at weeks 2 (d14) and 4 (d28) after priming. Combinations of route of immunization may be employed (e.g., s.c. prime, followed by i.n. immunization on day 14).

Five mice from each treatment group are sacrificed at day 35 after prime immunization (25 of the 75 mice). Serum is harvested before priming immunization (d0), at day 7, 28, and 56. In addition nasal lavage (NL) fluids or bronchoalveolar lavage (BAL) and splenocytes are harvested at sacrifice.

Antigen-specific antibody in the respiratory tract fluids (gathered by NL or BAL upon sacrifice), and in the serum are titred by serial dilution and application to ELISA, with immobilized whole recombinant antigen (or vaccinia proteins), peptide, non-relevant peptide control and serially diluted serum from all treatment groups, including naive mice. Isotype of specific antibodies is determined.

Neutralizing antibodies are present in mice immunized by either nasal or subcutaneous administration. The antibodies react with both antigenic peptide and whole viral protein. Nasal administration is more efficient to promote a mucosal immune response, while subcutaneous administration is more efficient to promote a systemic immune response against poxvirus.

### Example 4

### Alternative Methods of Preparing Immunoconjugates by the Dock-and-Lock (DNL) Technique

DDD and AD Fusion Proteins

The DNL technique can be used to make dimers, trimers, tetramers, hexamers, etc. comprising virtually any antibodies or fragments thereof or other protein or peptide moieties. For certain preferred embodiments, IgG antibodies, F(ab)₂ antibody fragments and subunit antigenic peptides, may be produced as fusion proteins containing either a dimerization and docking domain (DDD) or anchoring domain (AD) sequence. Although in preferred embodiments the DDD and AD moieties are produced as fusion proteins, the skilled artisan will realize that other methods of conjugation, such as chemical cross-linking, may be utilized within the scope of the claimed methods and compositions.

DNL constructs may be formed by combining, for example, an Fab-DDD fusion protein of an anti-HLA-DR or anti-CD74 antibody with a vIL18BP105-AD fusion protein. Alternatively, constructs may be made that combine IgG-AD fusion proteins with vIL18BP105-DDD fusion proteins. The technique is not limiting and any protein or peptide of use may be produced as an AD or DDD fusion protein for incorporation into a DNL construct. Where chemical cross-linking is utilized, the AD and DDD conjugates are not limited to proteins or peptides and may comprise any molecule that may be cross-linked to an AD or DDD sequence using any cross-linking technique known in the art.

Independent transgenic cell lines may be developed for each DDD or AD fusion protein. Once produced, the modules can be purified if desired or maintained in the cell culture supernatant fluid. Following production, any DDD-fusion protein module can be combined with any AD-fusion protein module to generate a DNL construct. For different types of constructs, different AD or DDD sequences may be utilized. Exemplary DDD and AD sequences are provided below.

(SEQ ID NO: 24)

| | |
|---|---|
| DDD1: | SHIQIPPGLTELLQGYTVEVLRQQPPDLVEFAVEYFTRLREARA |

(SEQ ID NO: 25)

| | |
|---|---|
| DDD2: | CGHIQIPPGLTELLQGYTVEVLRQQPPDLVEFAVEYFTRLREARA |

(SEQ ID NO: 26)

| | |
|---|---|
| AD1: | QIEYLAKQIVDNAIQQA |

(SEQ ID NO: 27)

| | |
|---|---|
| AD2: | CGQIEYLAKQIVDNAIQQAGC |

Expression Vectors

The plasmid vector pdHL2 has been used to produce a number of antibodies and antibody-based constructs. See Gillies et al., J Immunol Methods (1989), 125:191-202; Losman et al., Cancer (Phila) (1997), 80:2660-6. The di-cistronic mammalian expression vector directs the synthesis of the heavy and light chains of IgG. The vector sequences are mostly identical for many different IgG-pdHL2 constructs, with the only differences existing in the variable domain (VH and VL) sequences. Using molecular biology tools known to those skilled in the art, these IgG expression vectors can be converted into Fab-DDD or Fab-AD expression vectors. To generate Fab-DDD expression vectors, the coding sequences for the hinge, CH₂ and CH3 domains of the heavy chain are replaced with a sequence encoding the first 4 residues of the hinge, a 14 residue Gly-Ser linker and the first 44 residues of human RIIα (referred to as DDD1). To generate Fab-AD expression vectors, the sequences for the hinge, CH2 and CH3 domains of IgG are replaced with a sequence encoding the first 4 residues of the hinge, a 15 residue Gly-Ser linker and a 17 residue synthetic AD called AKAP-IS (referred to as AD1), which was generated using bioinformatics and peptide array technology and shown to bind RIIα dimers with a very high affinity (0.4 nM). See Alto, et al. Proc. Natl. Acad. Sci., U.S.A (2003), 100:4445-50.

Two shuttle vectors were designed to facilitate the conversion of IgG-pdHL2 vectors to either Fab-DDD1 or Fab-AD1 expression vectors. Using this technique, we have produced AD and/or DDD fusion proteins and encoding plasmids for Fab expression of a wide variety of known antibodies, such as hLL1, hLL2, hPAM4, hR1, hRS7, hMN-14, hMN-15, hA19, hA20 and many others.

Trimeric DNL construct are obtained by reacting a DDD fusion protein comprising, e.g., an IgG antibody or F(ab) antibody fragment with an AD fusion protein comprising, e.g., a subunit antigenic peptide, at a molar ratio of between 1.4:1 and 2:1. The total protein concentration is 1.5 mg/ml in PBS containing 1 mM EDTA. Subsequent steps may involve TCEP reduction, HIC chromatography, DMSO oxidation, and affinity chromatography to obtain the purified DNL construct. Addition of 5 mM TCEP rapidly results in the formation of a₂b complex. Binding assays show that the antibody moiety and antigenic peptide moieties retain their functional properties of respectively antigen-binding and antigenicity.

Using this technique, virtually any antibody or antibody fragment may be attached to any subunit antigenic peptide by preparing appropriate fusion proteins of each, comprising complementary DDD and AD moieties.

The following peptides are made as AD2 modules incorporating a linking sequence attaching a subunit vaccine peptide. The AD2-peptide fusion is combined with DDD2-linked IgG or Fab moieties to provide a subunit based vaccine incorporating an APC-targeting antibody or antibody fragment.

ND8L
(SEQ ID NO: 34)
SIFLQVLDHKNVYFQGGGSCGQIEYLAKQIVDNAIQQAGC
CD8L
(SEQ ID NO: 35)
CGQIEYLAKQIVDNAIQQAGCGGGSSIFLQVLDHKNVYFQ
NIL18BP
(SEQ ID NO: 36)
HYRFTCVLTTLNGVSGGGSCGQIEYLAKQIVDNAIQQAGC
C1L18BP
(SEQ ID NO: 37)
CGQIEYLAKQIVDNAIQQAGCGGGSHYRFTCVLTTLNGVS
CSCRD8L
(SEQ ID NO: 38)
CGQIEYLAKQIVDNAIQQAGCGGGSYHQFVIDQLKLSVNF
CSCR1L18BP105
(SEQ ID NO: 39)
CGQIEYLAKQIVDNAIQQAGCGGGSGNCTFVTYLRHLSTV

### Example 5

### Liposome Formulation for Nasal Administration of Subunit Based Vaccine

A liposome formulation of antigenic peptide conjugated to L243 antibody was prepared by standard techniques. The intranasal peptides were designed with linkers at both the C-terminal and N-terminal ends. The C-terminal linker was used for conjugation of the L243 antibody. The N-terminal linker was used to facilitate attachment to the liposome, via palmitoylation. The peptide conjugates were as indicated below. The CD8L118 peptide was not a lipoprotein and was encapsulated into liposomes.

L1R183

| | |
|---|---|
| GVQFYMIVIGVIILAALF | (SEQ ID NO: 40) |
| Conjugated L1R183 | |
| KKKKGVQFYMIVIGVIILAALFPSEC | (SEQ ID NO: 41) |
| Conjugated A27L3 | |
| KSGTLFPGDDDLAIPATEFFSTKAAKKPSEC | (SEQ ID NO: 42) |
| Conjugated IL18BP105 | |
| KSHYRFTCVLTTLNGVSPESC | (SEQ ID NO: 43) |
| CD8L118 | |
| HDDGLIIISIFLQVLDHKNVYFQKIGGGSC | (SEQ ID NO: 44) |

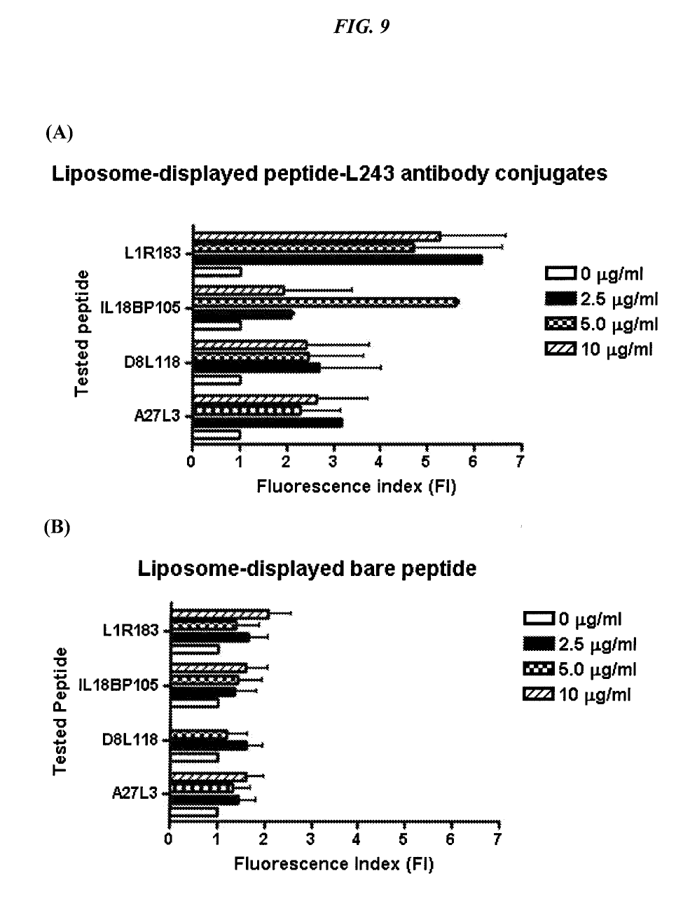

FIG. 9(A) shows the results of nasal administration of a liposome formulated subunit vaccine. Peptides were prepared and conjugated to antibody as described in Examples 1 and 2 above. The results presented in FIG. 9 show T cell proliferation in response to incubation with the designated peptide in vitro after nasal immunization of mice. The strongest effect on T cell proliferation (FIG. 9A) was observed with the L1R183 antigenic peptide (SEQ ID NO:39) derived from the L1R antigen, an immunodominant intracellular mature virion (IMV) protein that offers post-exposure prophylaxis. Immunization of mice with liposome-displayed bare peptide alone (FIG. 9B) produced little effect on T cell proliferation, regardless of the tested peptide. Immunization with free peptide in the absence of liposome or with empty liposomes also had little to no effect on T cell proliferation (data not shown). The results demonstrate that nasal administration of a subunit based poxvirus vaccine, using conjugation to an APC targeting antibody, is effective to induce an immune response.

All of the COMPOSITIONS and METHODS disclosed and claimed herein can be made and used without undue experimentation in light of the present disclosure. While the compositions and methods have been described in terms of preferred embodiments, it is apparent to those of skill in the art that variations maybe applied to the COMPOSITIONS and METHODS and in the steps or in the sequence of steps of the METHODS described herein without departing from the concept, spirit and scope of the invention. More specifically, certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. An immunoconjugate comprising:
a) at least one antigenic peptide from a poxvirus protein; and
b) an antibody or antigen-binding fragment thereof that binds to an antigen-presenting cell (APC), wherein the antibody or fragment is conjugated to the antigenic peptide;
wherein administration of the immunoconjugate to a subject induces an immune response against the poxvirus.

2. The immunoconjugate of claim 1, wherein the poxvirus protein is a viral immunomodulating factor.

3. The immunoconjugate of claim 2, wherein the poxvirus protein is a viral IL-18 binding protein (vIL18BP).

4. The immunoconjugate of claim 1, wherein the poxvirus protein is an envelope protein.

5. The immunoconjugate of claim 1, wherein the poxvirus protein is selected from the group consisting of L1R, A27L and D8L.

6. The immunoconjugate of claim 1, wherein the immunoconjugate comprises at least one antigenic peptide from a viral immunomodulating factor and at least one antigenic peptide from a viral envelope protein.

7. The immunoconjugate of claim 1, wherein the poxvirus is smallpox.

8. The immunoconjugate of claim 1, wherein the antibody or fragment thereof binds to an APC antigen selected from the group consisting of HLA-DR, CD74, CD209 (DC-SIGN), CD34, CD74, CD205, TLR 2 (toll-like receptor 2), TLR 4, TLR 7, TLR 9, BDCA-2, BDCA-3 and BDCA-4.

9. The immunoconjugate of claim 1, wherein the APC antigen is HLA-DR or CD74.

10. The immunoconjugate of claim 9, wherein the antibody or fragment thereof is an anti-HLA-DR antibody comprising the heavy chain complementarity determining region (CDR) sequences CDR1 NYGMN (SEQ ID NO:1), CDR2 WINTYTREPTYADDFKG (SEQ ID NO:2), and CDR3 DITAVVPTGFDY (SEQ ID NO:3) and the light chain CDR sequences CDR1 RASENIYSNLA(SEQ ID NO:4), CDR2 AASNLAD (SEQ ID NO:5), and CDR3 QHFWTTPWA (SEQ ID NO:6).

11. The immunoconjugate of claim 9, wherein the antibody or fragment thereof is an anti-CD74 antibody comprising the light chain CDR sequences CDR1 RSSQSLVHRNGNTYLH (SEQ ID NO:7), CDR2 TVSNRFS (SEQ ID NO:8), and CDR3 SQSSHVPPT (SEQ ID NO:9) and the heavy chain CDR sequences CDR1 NYGVN (SEQ ID NO:10), CDR2 WINPNTGEPTFDDDFKG (SEQ ID NO:11), and CDR3SRGKNEAWFAY (SEQ ID NO:12).

12. The immunoconjugate of claim 1, wherein the antigenic peptide has an amino acid sequence selected from the group consisting of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39 and SEQ ID NO:40.

13. The immunoconjugate of claim 1, wherein the immunoconjugate is a fusion protein comprising the antigenic peptide and the antibody or antibody fragment.

14. The immunoconjugate of claim 1, wherein the antigenic peptide is covalently attached to the antibody or antibody fragment.

15. The immunoconjugate of claim 1, wherein the antigenic peptide is part of a first fusion protein, the antibody or fragment thereof is part of a second fusion protein, and the first and second fusion proteins bind to each other.

16. The immunoconjugate of claim 15, wherein first fusion protein comprises a dimerization and docking domain (DDD) moiety from human protein kinase A (PKA) RIα, RIIα, RIβ or RII β and the second fusion protein comprises an anchoring domain from an AKAP protein.

17. A pharmaceutical composition comprising an immunoconjugate according to claim 1 and a pharmaceutically acceptable carrier.

18. The pharmaceutical composition of claim 17, wherein the pharmaceutical composition is a subunit vaccine.

19. The pharmaceutical composition of claim 18, wherein administration of the vaccine to a subject provides immunity to poxvirus infection.

20. The pharmaceutical composition of claim 18, wherein administration of the vaccine to a subject induces immunity to smallpox infection.

21. The pharmaceutical composition of claim 18, wherein the composition further comprises at least one adjuvant.

22. A method of inducing immunity to poxvirus infection comprising administering to a subject a subunit vaccine according to claim 18.

23. The method of claim 22, wherein the poxvirus is smallpox.

24. The method of claim 22, wherein the composition is administered subcutaneously or nasally.

25. The method of claim 24, wherein a liposome subunit vaccine is administered nasally.

26. The method of claim 17, wherein the immunoconjugate is administered in the form of an expression vector that encodes a fusion protein comprising at least one antigenic peptide from a poxvirus protein and an antibody or antigen-binding fragment thereof that binds to an APC.
